(19) United States
(12) Patent Application Publication
(10) Pub. No.: US 2022/0362327 A1
(43) Pub. Date: Nov. 17, 2022

(54) COMPOSITIONS AND METHODS FOR WOUND HEALING

(71) Applicant: Nasser DIAB, Bridgeview, IL (US)

(72) Inventor: Nasser DIAB, Bridgeview, IL (US)

(21) Appl. No.: 17/319,691

(22) Filed: May 13, 2021

(51) Int. Cl. A61K 38/12; A61K 9/00; A61P 17/02; A61K 38/48; A61K 31/496; A61K 31/431

(52) U.S. Cl. CPC A61K 38/12; A61K 9/0014; A61P 17/02; A61K 38/4886; A61K 31/496; A61K 31/431; A61K 9/06

(57) ABSTRACT

Featured are compositions and methods for wound healing.

### BACKGROUND OF THE INVENTION

Skin ulcerations, such as diabetic ulcers, can lead to significant medical complications in patients. In fact, statistics show that 85% of lower limb amputations in patients with diabetes are preceded by ulceration and that 15% of diabetic foot ulcers result in lower extremity amputation (Ramsey et al. Diabetes Care 22:382, 1999). Furthermore, a 2018 retrospective analysis of Medicare cost estimates for all wounds ranged from $28.1 billion to $96.8 billion, including costs for infection management, among which surgical wounds and diabetic ulcers were the most expensive to treat (Sen, C. K. Advances in Wound Care 8:39, 2019). Wounds such as diabetic ulcers and surgical wounds may be infected by bacteria, such as Staphylococcus, Streptococcus, and Pseudomonas, complicating and delaying or preventing the healing process.

Therefore, new methodologies in wound treatment are needed to reduce pain and suffering, prevent surgical debridement and/or unnecessary amputations, and/or to expedite healing.

### SUMMARY OF THE INVENTION

The invention features compositions and methods for wound healing. In a first aspect, the invention features a topical composition for treating a wound in a subject comprising at least first and second layers, wherein: (a) the first layer comprises a powder, wherein the powder comprises 25-95% by total weight bacitracin zinc-polymyxin B sulfate powder comprising 100-1,000 units bacitracin per gram and 5,000-15,000 units polymyxin B per gram and 5-75% (e.g., 5-25% or 10-20%) by total weight ciprofloxacin; and (b) the second layer comprises an ointment, wherein the ointment comprises: (i) an admixture comprising 40-80% by total weight collagenase ointment comprising 100-500 units collagenase per gram, 4-50% by total weight ciprofloxacin powder, and 20-50% by total weight bacitracin zinc-polymyxin B sulfate powder comprising 100-1,000 units bacitracin per gram and 5,000-15,000 units polymyxin B per gram; or (ii) an admixture comprising 40-80% by total weight collagenase ointment comprising 100-500 units collagenase per gram, and 20-60% by total weight bacitracin zinc-polymyxin B sulfate powder comprising 100-1,000 units bacitracin per gram and 5,000-15,000 units polymyxin B per gram.

In some embodiments, the first and/or second layer further comprises 4-30% by total weight methicillin sodium powder.

In some embodiments, the first and/or second layer further comprises 4-30% by total weight ceftaroline fosamil powder.

In some embodiments, the admixture of (b)(ii) further comprises 4-30% by total weight ceftazidime sodium carbonate dry powder comprising 110-120 mg sodium carbonate per gram of ceftazidime.

In an embodiment, the first layer comprises a weight of about 4 g to about 20 g (e.g., about 8 g to about 15 g) and/or the second layer comprises a weight of about 4 g to about 30 g (e.g., about 5 g to about 25 g, such as about 16-18 g).

In a second aspect, the invention features a method for treating a wound in a subject (e.g., a human) comprising topically applying a composition described herein to the wound (e.g., a composition of the first aspect), in which the first layer of the composition is applied to the wound and the second layer of the composition is applied over the first layer. In an embodiment, the first layer comprises a powder comprising 25-95% by total weight bacitracin zinc-polymyxin B sulfate powder comprising 100-1,000 units bacitracin per gram and 5,000-15,000 units polymyxin B per gram and 5-75% (e.g., 5-25% or 10-20%) by total weight ciprofloxacin; and the second layer comprises an ointment comprising: (i) an admixture comprising 40-80% by total weight collagenase ointment comprising 100-500 units collagenase per gram, 4-50% by total weight ciprofloxacin powder, and 20-50% by total weight bacitracin zinc-polymyxin B sulfate powder comprising 100-1,000 units bacitracin per gram and 5,000-15,000 units polymyxin B per gram; or (ii) an admixture comprising 40-80% by total weight collagenase ointment comprising 100-500 units collagenase per gram, and 20-60% by total weight bacitracin zinc-polymyxin B sulfate powder comprising 100-1,000 units bacitracin per gram and 5,000-15,000 units polymyxin B per gram. In some embodiments, the first and/or second layer further comprises 4-30% by total weight methicillin sodium powder. In some embodiments, the first and/or second layer further comprises 4-30% by total weight ceftaroline fosamil powder. In some embodiments, the second layer comprising the admixture of (ii) further comprises 4-30% by total weight ceftazidime sodium carbonate dry powder comprising 110-120 mg sodium carbonate per gram of ceftazidime.

In an embodiment, the first layer of the composition comprises a weight of about 4 g to about 20 g (e.g., about 8 g to about 15 g) and/or the second layer of the composition comprises a weight of about 4 g to about 30 g (e.g., about 5 g to about 25 g, such as about 16-18 g).

In some embodiments, prior to application of the composition, the wound is cleaned with 0.05-0.10% normal saline (NS).

In some embodiments, the second layer comprises a gauze that is impregnated with the ointment, and wherein the gauze is applied over the first layer. In some embodiments, the method further comprises covering the gauze with an additional layer of gauze, KERLIX™, and/or a wound pad. In some embodiments, the additional layer of gauze, KERLIX™, and/or wound pad is secured to the subject with tape.

In some embodiments, the wound is an external ulcer or a pressure ulcer, a surgical wound, is caused by or results from diabetes or systemic lupus erythematosus (SLE), or is infected, or wherein the wound comprises gangrene. In some embodiments, the external ulcer is a diabetic foot ulcer or the pressure ulcer is a bed sore. In some embodiments, the infected wound comprises methicillin-resistant Staphylococcus aureus (MRSA) or Pseudomonas aeruginosa.

In some embodiments, the method comprises topically applying the composition to the wound one or more times daily, weekly, biweekly, or monthly. In some embodiments, the topical application occurs one or more times every one, two, three, four, five, six, or seven days.

In some embodiments, the topical application occurs for 1 to 52 weeks or more.

In some embodiments, the first and/or second layer of the composition further comprises 4-30% by total weight methicillin sodium powder and/or 4-30% by total weight ceftaroline fosamil powder.

In some embodiments, the admixture of (b)(ii) further comprises 4-30% by total weight ceftazidime sodium carbonate dry powder comprising 110-120 mg sodium carbonate per gram of ceftazidime.

### Definitions

As used herein, the term “about” means that the amount or value in question can be the value designated ±10% variation unless otherwise indicated or inferred. The term is intended to convey that similar values promote equivalent results or effects recited in the claims.

A weight percent (e.g., “wt. %,” or “% by total weight”) of a component, unless specifically stated to the contrary, is based on the total weight of the formulation or composition in which the component is included.

As used herein, the term “subject” can be a vertebrate, such as a mammal (e.g., a human). The term does not denote a particular age or sex. Thus, adult, adolescent, and newborn subjects, as well as male and female subjects, are intended to be covered. A patient refers to a subject afflicted with an ailment, disease, or disorder, such as a skin ulceration.

As used herein, the term “treatment” (and grammatical variations thereof such as “treat” or “treating”) refers to the medical management of a patient with the intent to cure, ameliorate, stabilize, or prevent an ailment, disease, pathological condition, or disorder, such as a skin ulceration. This term includes active treatment, that is, treatment directed specifically toward the improvement of a skin ailment, disease, pathological condition, or disorder, and also includes causal treatment, that is, treatment directed toward removal of the cause of the associated skin ailment, disease, pathological condition, or disorder. In addition, this term includes palliative treatment, that is, treatment designed for the relief of symptoms rather than the curing of the skin ailment, disease, pathological condition, or disorder; preventative treatment, that is, treatment directed to minimizing or partially or completely inhibiting the development of the associated skin ailment, disease, pathological condition, or disorder; and supportive treatment, that is, treatment employed to supplement another specific therapy directed toward the improvement of the associated skin ailment, disease, pathological condition, or disorder. In various aspects, the term covers any treatment of a subject, including a mammal (e.g., a human), and includes: (i) preventing the skin ailment from occurring in a subject that can be predisposed to the skin ailment but has not yet been diagnosed as having it; (ii) inhibiting the skin ailment, i.e., arresting its development; or (iii) relieving the skin ailment, i.e., causing regression of the skin ailment.

As used herein, the term “mixture” means a material made of two or more different substances that are combined. In some cases, a mixture described herein can be a homogenous mixture of the two or more different substances, e.g., the mixture can have the same proportions of its components (e.g., the two or more substances) throughout any given sample of the mixture. In some cases, a mixture as provided herein can be a heterogeneous mixture of the two or more different substances, e.g., the proportions of the components of the mixture (e.g., the two or more substances) can vary throughout the mixture. In some cases, a mixture is a liquid solution, e.g., the mixture is present in liquid phase. In some instances, a liquid solution can be regarded as comprising a liquid solvent and a solute. Mixing a solute in a liquid solvent can be termed as “dissolution” process. In some cases, a liquid solution is a liquid-in-liquid solution (e.g., a liquid solute dissolved in a liquid solvent), a solid-in-liquid solution (e.g., a solid solute dissolved in a liquid solvent), or a gas-in-liquid solution (e.g., a solid solute dissolved in a liquid solvent). In some cases, there is more than one solvent and/or more than one solute. In some cases, a mixture is a colloid, liquid suspension, or emulsion. In some cases, a mixture is a solid mixture, e.g., the mixture is present in solid phase.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compositions or methods disclosed herein.

As used herein, the terms “administering” and “administration” refer to any method of providing a pharmaceutical preparation to a subject. Such methods are well known to those skilled in the art and include, but are not limited to, topical administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “topical composition” refers to a pharmaceutical composition that is administered or delivered externally to the skin or an epithelial layer through non-invasive means, e.g., direct application or application of the pharmaceutical composition to gauze, which is then applied to the skin. As used herein, the term “topical treatment,” “topical administration,” “topical delivery,” or “topical application” refers to the delivery of a substance to skin or an epithelial layer accessible through non-invasive means, e.g., direct application or application of the pharmaceutical composition to gauze, which is then applied to the skin. Topical application of a composition can produce a local pharmacodynamic effect on the subject, e.g., the topically applied composition has a pharmacodynamic effect at or proximate to the particular part of the body (e.g. skin) where the composition is delivered. In some other embodiments, topical application of a composition as discussed herein is used only to refer to the delivery mode (locally to, e.g., a specific surface area), whereas the composition can have either a local or systemic pharmacodynamic effect. For instance, the composition can either stay local at or proximate to the administration site, or can enter a circulation system (e.g., blood or lymphoid system) of the subject body, through which the composition can be transported to remote parts of the body that are typically not reachable by the composition via routes other than the circulation systems.

As used herein, the term “wound” or “open wound” refers to an injury, especially one in which the skin or another external surface is torn, pierced, cut, or otherwise broken, or in which the integrity of the epidermal layer and/or the dermal layer is compromised. This may include, but is not limited to, abrasion, laceration, puncture, avulsion, burn, and incision of the skin. As used herein, a “surgical wound” is a cut or incision in the skin that is usually made by a scalpel or surgical tool during surgery. As used herein, an “infected wound” or “infection” refers to a wound that has drainage, pus, inflammation, necrotizing fasciitis, and/or foul odor. Additionally, the patient may develop fever, and cellulitis as a result of infection of a wound. A wound(s) may be caused by an accident with a sharp object, a car accident, rubbing skin or scraping against a rough or hard surface (e.g., road rash), a burn, pressure (acute or chronic), or a gunshot. The term “wound bed” refers to the base or floor of an injury (e.g., burn, laceration, ulcer, abrasion, avulsion, or incision).

As used herein, the term “ulcer” is a site of damage to the skin or mucous membrane that is often characterized by the formation of pus, death of tissue, and is frequently accompanied by an inflammatory reaction. An “external ulcer” refers to a skin ulcer. A “pressure ulcer” or “pressure sore” refers to an ulcer that develops because of pressure over a bony prominence. A “bed sore” or “decubitus ulcer” refers to a pressure ulcer that occurs from lying in bed, sitting in a wheelchair, or wearing a cast for a prolonged time. A “foot ulcer” refers to an ulcer of the foot.

As used herein, a “diabetic wound” is a wound that associated with diabetes and elevated blood sugar. Elevated blood sugar may prevent nutrients and oxygen from energizing cells, prevent the immune system of a subject with diabetes from functioning efficiently, and may increase inflammation of cells. These factors may cause a wound to heal slowly and be more susceptible to complications like further injury (e.g., abrasion, bruising) or infection. A “diabetic ulcer” is an ulcer that is associated with the aforementioned effects of diabetes and elevated blood sugar.

As used herein, the term “powder” is a fine, dry particle produced by the grinding, crushing, or disintegration of a solid substance. As used herein, the term “paste,” “medicated paste,” “ointment,” or “medicated ointment” refers to a pharmaceutical composition that is a smooth, soft, moist, and/or oily preparation that may be applied topically to the skin for medicinal purposes. The “paste” or “ointment” may also be applied to a sterile wound dressing, such as gauze, which is then applied to the skin so the paste or ointment contacts a wound or ulcer to be treated.

As used herein, the term “maturation” refers to the process of type III collagen converting to type I collagen. A wound is matured when the type III collagen has fully remodeled to type I, and the wound fully closes. The term “approximation” refers to the edges of the wound contracting together, allowing the wound to close easily.

### DETAILED DESCRIPTION

Disclosed are improved compositions and methods for healing wounds, in particular those occurring on the skin. This disclosure relates generally to topical compositions, preparations, and methods of healing a wound(s) in a subject (e.g., a human). Topical compositions and methods of treatment disclosed may be used to treat skin wounds and external ulcers comprising pressure ulcers, bed sores (e.g., decubitus), foot ulcers, diabetic foot ulcers, surgical wounds, and ulcers caused by gas gangrene. Topical compositions disclosed may be used to treat pressure ulcers at any of the four stages, in which stage 1 corresponds to skin that is fully intact with non-blanchable erythema (usually in a Caucasian subject), or, dyschromia (usually in a non-Caucasian subject) present; stage 2 corresponds to a deeper skin injury that disrupts the skin barrier function, that is, the first layer of the epidermis, e.g., the papillary layer; stage 3 corresponds to a full thickness injury, i.e., full destruction of the papillary and the reticular layers of the dermis, but the subcutaneous tissue as represented by the fibrous tissue, remains intact; and stage 4 corresponds to a deep, full thickness tissue loss of the papillary, reticular, and the subcutaneous tissue, and loss of tissue that could be bone, tendon, muscle, ligament and/or, metal if the subject has a surgical implant.

Topical compositions and methods of treatment disclosed herein may be used to treat diabetic wounds or diabetic ulcers. For example, the topical compositions and methods of treatment disclosed herein may be used to treat diabetic ulcers that are inflamed or infected. In some cases, the topical compositions and methods of treatment disclosed herein may be used to prevent or treat infection of a wound or an ulcer. For example, topical compositions and methods of treatment disclosed herein may be used to prevent or treat an infection of an ulcer or wound caused by bacteria such as, but not limited to, Staphylococcus (e.g., Staphylococcus aureus, such as methicillin-resistant Staphylococcus aureus (MRSA)), Streptococcus, and/or Pseudomonas (e.g., Pseudomonas aeruginosa). In other examples, the topical compositions and methods of treatment disclosed herein may prevent or reduce the need for surgical debridement or amputation. Topical compositions and methods of treatment disclosed herein may be used to heal ulceration due to, e.g., an autoimmune condition, such diabetes and systemic lupus erythematosus. Topical compositions and methods of treatment disclosed herein may be used to heal ulceration caused by, for example, gas gangrene. In many examples, the topical compositions and methods of treatment disclosed herein may reduce the time of treatment and increase the healing time, as compared with other methods of wound healing known in the art.

### Compositions

A topical composition of the disclosure may be formulated as a paste or ointment which may be applied to the wound by laying the formulated paste on sterile gauze. The paste impregnated gauze may then be placed on the wound so that the medicated paste is in contact with the wound and wound edges. A topical composition of the disclosure may be formulated as a mixture of antibiotics which may be applied or sprayed in a wound, covering the wound bed and wound edges. The volume of the topical composition will depend on the size of a wound. The composition can be formulated in a single layer or multiple layers (e.g., a first and a second layer). The layers can be the same or different.

In an example, the first layer of the composition can be a powder containing about 25-95% by total weight bacitracin zinc-polymyxin B sulfate powder comprising 100-1,000 units bacitracin per gram and about 5,000-15,000 units polymyxin B per gram and about 5-75% (e.g., 5-25% or 10-20%) by total weight ciprofloxacin. The first layer can be prepared with a total weight of about 4 g to about 20 g (e.g., about 8 g to about 15 g). The second layer of the composition can be an ointment containing: (i) an admixture comprising about 40-80% by total weight collagenase ointment comprising 100-500 units collagenase per gram, about 4-50% by total weight ciprofloxacin powder, and about 20-50% by total weight bacitracin zinc-polymyxin B sulfate powder comprising 100-1,000 units bacitracin per gram and about 5,000-15,000 units polymyxin B per gram; or (ii) an admixture comprising about 40-80% by total weight collagenase ointment comprising 100-500 units collagenase per gram, and about 20-60% by total weight bacitracin zinc-polymyxin B sulfate powder comprising 100-1,000 units bacitracin per gram and about 5,000-15,000 units polymyxin B per gram. The second layer of the composition can be prepared with a total weight of about 4 g to about 30 g (e.g., about 5 g to about 25 g, such as about 16-18 g).

A topical composition of the disclosure may be a paste or ointment containing collagenase ointment, ciprofloxacin (or a salt thereof), and bacitracin zinc-polymyxin B sulfate (e.g., NEOSPORIN®). For example, the topical composition may contain 40-80% by total weight collagenase ointment (e.g., 40%, 45%, 50%, 55%, 60%, 65%, 70%, 75%, or 80%). The collagenase ointment can contain, for example, 100-500 units of collagenase per gram. The topical composition may also contain 4-50% by total weight ciprofloxacin (e.g., 4%, 5%, 6%, 7%, 8%, 9%, 10%, 15%, 20%, 25%, 30%, 35%, 40%, 45%, or 50%). Finally, the topical composition may contain 20-50% by total weight bacitracin zinc-polymyxin B sulfate (e.g., 20%, 25%, 30%, 35%, 40%, 45%, or 50%). The bacitracin zinc-polymyxin B sulfate can contain, for example, 100-1,000 units of bacitracin per gram and 5,000-15,000 units of polymyxin B per gram.

A topical composition of the disclosure may be a paste or ointment containing collagenase ointment and bacitracin zinc-polymyxin B sulfate. For example, the topical composition may contain 40-80% by total weight collagenase ointment (e.g., 40%, 45%, 50%, 55%, 60%, 65%, 70%, 75%, or 80%). The collagenase ointment can contain, for example, 100-500 units of collagenase per gram. The topical composition may also contain 20-60% by total weight bacitracin zinc-polymyxin B sulfate (e.g., 20%, 25%, 30%, 35%, 40%, 45%, 50%, 55%, or 60%). The bacitracin zinc-polymyxin B sulfate can contain, for example, 100-1,000 units of bacitracin per gram and 5,000-15,000 units of polymyxin B per gram.

A topical composition of the disclosure may be a mixture of antibiotics containing bacitracin zinc-polymyxin B sulfate and ciprofloxacin, or a salt thereof. For example, the topical composition may contain 25-95% by total weight bacitracin zinc-polymyxin B sulfate (e.g., 25%, 30%, 35%, 40%, 45%, 50%, 55%, 60%, 65%, 70%, 75%, 80%, 85%, 90%, 95%). The bacitracin zinc-polymyxin B sulfate can contain, for example, 100-1,000 units bacitracin per gram and 5,000-15,000 units polymyxin B per gram. The topical composition may also contain 5-75% by total weight ciprofloxacin (e.g., 5%, 10%, 15%, 20%, 25%, 30%, 35%, 40%, 45%, 50%, 55%, 60%, 65%, 70%, or 75%).

A topical composition of the disclosure may be a paste or ointment containing collagenase ointment, ciprofloxacin (or a salt thereof), bacitracin zinc-polymyxin B sulfate, and methicillin sodium powder (e.g., Cas. No. 132-92-3; see, e.g., Cat. No. M62800-5.0, Research Products, International (RPI), Mount Prospect, Ill.). For example, the topical composition may contain 40-80% by total weight collagenase ointment (e.g., 40%, 45%, 50%, 55%, 60%, 65%, 70%, 75%, or 80%). The collagenase ointment can contain, for example, 100-500 units collagenase per gram. The topical composition may also contain 4-50% by total weight ciprofloxacin (e.g., 4%, 5%, 6%, 7%, 8%, 9%, 10%, 15%, 20%, 25%, 30%, 35%, 40%, 45%, or 50%). The topical composition may also contain 20-50% by total weight bacitracin zinc-polymyxin B sulfate (e.g., 20%, 25%, 30%, 35%, 40%, 45%, or 50%). The bacitracin zinc-polymyxin B sulfate can contain, for example, 100-1,000 units bacitracin per gram and 5,000-15,000 units polymyxin B per gram. Finally, the topical composition may also contain 4-30% by total weight methicillin sodium powder (e.g., 4%, 5%, 6%, 7%, 8%, 9%, 10%, 15%, 20%, 25%, or 30%).

A topical composition of the disclosure may be a paste or ointment containing collagenase ointment, bacitracin zinc-polymyxin B sulfate, and methicillin sodium powder. For example, the topical composition may contain 40-80% by total weight collagenase ointment (e.g., 40%, 45%, 50%, 55%, 60%, 65%, 70%, 75%, or 80%). The collagenase ointment can contain, for example, 100-500 units collagenase per gram. The topical composition may also contain 20-50% by total weight bacitracin zinc-polymyxin B sulfate (e.g., 20%, 25%, 30%, 35%, 40%, 45%, or 50%). The bacitracin zinc-polymyxin B sulfate can contain, for example, 100-1,000 units bacitracin per gram and 5,000-15,000 units polymyxin B per gram. Finally, the topical composition may also contain 4-30% by total weight methicillin sodium powder (e.g., 4%, 5%, 6%, 7%, 8%, 9%, 10%, 15%, 20%, 25%, or 30%).

A topical composition of the disclosure may be a mixture of antibiotics containing bacitracin zinc-polymyxin B sulfate, ciprofloxacin (or a salt thereof), and methicillin sodium powder. For example, the topical composition may contain 20-50% by total weight bacitracin zinc-polymyxin B sulfate (e.g., 20%, 25%, 30%, 35%, 40%, 45%, or 50%). The bacitracin zinc-polymyxin B sulfate can contain, for example, 100-1,000 units bacitracin per gram and 5,000-15,000 units polymyxin B per gram. The topical composition may also contain 5-50% by total weight ciprofloxacin (e.g., 5%, 10%, 15%, 20%, 25%, 30%, 35%, 40%, 45%, or 50%). Finally, the topical composition may also contain 4-30% by total weight methicillin sodium powder (e.g., 4%, 5%, 6%, 7%, 8%, 9%, 10%, 15%, 20%, 25%, or 30%).

A topical composition of the disclosure may be a paste or ointment containing collagenase ointment, ciprofloxacin (or a salt thereof), bacitracin zinc-polymyxin B sulfate, and ceftaroline fosamil powder. For example, the topical composition may contain 40-80% by total weight collagenase ointment (e.g., 40%, 45%, 50%, 55%, 60%, 65%, 70%, 75%, or 80%). The collagenase ointment can contain, for example, 100-500 units collagenase per gram. The topical composition may also contain 4-50% by total weight ciprofloxacin (e.g., 4%, 5%, 6%, 7%, 8%, 9%, 10%, 15%, 20%, 25%, 30%, 35%, 40%, 45%, or 50%). The topical composition may also contain 20-50% by total weight bacitracin zinc-polymyxin B sulfate (e.g., 20%, 25%, 30%, 35%, 40%, 45%, or 50%). The bacitracin zinc-polymyxin B sulfate can contain, for example, 100-1,000 units bacitracin per gram and 5,000-15,000 units polymyxin B per gram. Finally, the topical composition may contain 4-30% by total weight ceftaroline fosamil powder (e.g., 4%, 5%, 6%, 7%, 8%, 9%, 10%, 15%, 20%, 25%, or 30%).

A topical composition of the disclosure may be a paste or ointment containing a mixture of collagenase ointment, bacitracin zinc-polymyxin B sulfate, and ceftaroline fosamil powder. For example, the topical composition may contain 40-80% by total weight collagenase ointment (e.g., 40%, 45%, 50%, 55%, 60%, 65%, 70%, 75%, or 80%). The collagenase ointment can contain, for example, 100-500 units collagenase per gram. The topical composition may also contain 20-50% by total weight bacitracin zinc-polymyxin B sulfate (e.g., 20%, 25%, 30%, 35%, 40%, 45%, or 50%). The bacitracin zinc-polymyxin B sulfate can contain, for example, 100-1,000 units bacitracin per gram and 5,000-15,000 units polymyxin B per gram. Finally, the topical composition may contain 4-30% by total weight ceftaroline fosamil powder (e.g., 4%, 5%, 6%, 7%, 8%, 9%, 10%, 15%, 20%, 25%, or 30%).

A topical composition of the disclosure may be a mixture of antibiotics containing bacitracin zinc-polymyxin B sulfate, ciprofloxacin (or a salt thereof), and ceftaroline fosamil powder. For example, the topical composition may contain 20-90% by total weight bacitracin zinc-polymyxin B sulfate (e.g., 20%, 25%, 30%, 35%, 40%, 45%, 50%, 55%, 60%, 65%, 70%, 75%, 80%, 85%, or 90%). The bacitracin zinc-polymyxin B sulfate can contain, for example, 100-1,000 units bacitracin per gram and 5,000-15,000 units polymyxin B per gram. The topical composition may also contain 5-50% by total weight ciprofloxacin (e.g., 5%, 10%, 15%, 20%, 25%, 30%, 35%, 40%, 45%, or 50%). Finally, the topical composition may contain 4-30% by total weight anhydrous ceftaroline fosamil powder (e.g., 4%, 5%, 6%, 7%, 8%, 9%, 10%, 15%, 20%, 25%, or 30%).

A topical composition of the disclosure may be a paste or ointment containing collagenase ointment, bacitracin zinc-polymyxin B sulfate, and ceftazidime sodium carbonate dry powder. For example, the topical composition may contain 40-80% by total weight collagenase ointment (e.g., 40%, 45%, 50%, 55%, 60%, 65%, 70%, 75%, or 80%). The collagenase ointment can contain, for example, 100-500 units collagenase per gram. The topical composition may also contain 20-50% by total weight bacitracin zinc-polymyxin B sulfate (e.g., 20%, 25%, 30%, 35%, 40%, 45%, or 50%). The bacitracin zinc-polymyxin B sulfate can contain, for example, 100-1,000 units bacitracin per gram and 5,000-15,000 units polymyxin B per gram. Finally, the topical composition may contain 4-30% by total weight ceftazidime sodium carbonate dry powder (e.g., 4%, 5%, 6%, 7%, 8%, 9%, 10%, 15%, 20%, 25%, or 30%). The ceftazidime sodium carbonate dry powder can contain, for example, 110-120 mg sodium carbonate per gram of ceftazidime.

### Method of Formulating the Compositions

A topical composition of the disclosure may be formulated as a paste or ointment, containing collagenase ointment and antibiotics, or as a mixture of antibiotics. Any one of the topical compositions containing collagenase ointment, listed in the section above, may be formulated as a paste or an ointment according to the following procedure. First, the antibiotic(s) is ground into a powder if the antibiotic(s) is not already in powder form. For example, if ciprofloxacin is not already in powder form, a pill(s) made of ciprofloxacin (e.g., 100 mg, 250 mg, or 500 mg tablet(s), e.g., 2 to 2.5×500 mg tablets) may be placed in a mortar and pestle, then crushed and ground until a fine powder is formed. The ciprofloxacin may also be crushed, ground, milled, or formed by any other suitable method, such as, but not limited to, milling. The paste/ointment is then prepared by adding collagenase ointment and the antibiotic(s) in an appropriate, sterile container, such as a sterile bowl. The components are then mixed together using an appropriate, sterile mixing tool, such as a sterile wooden applicator stick, a sterile tongue depressor, a sterile spoon, a sterile stirring rod, or a sterile pestle. The topical composition can be prepared in with a total weight, including all added components, of about 4 g to about 30 g.

Any one of the topical compositions that do not contain collagenase ointment (e.g., a mixture of antibiotics) may be formulated according to the following procedure. First, the antibiotic(s) is ground into a powder, unless the antibiotic(s) is already in powder form. For example, if ciprofloxacin is not already in powder form, a pill made of ciprofloxacin (e.g., 100 mg, 250 mg, or 500 mg tablet(s), e.g., 2 to 2.5×500 mg tablets) may be placed in a mortar and pestle, then crushed and ground until a fine powder is achieved. The ciprofloxacin may also be crushed, ground, milled, or formed by any other suitable method such as, but not limited to, milling. Once the antibiotics are in powder form, the antibiotics are then added to an appropriate, sterile container, such as a sterile bowl. The components are then mixed together using an appropriate, sterile mixing tool, such as, but not limited to, a sterile whisk, a sterile wooden applicator stick, a sterile tongue depressor, a sterile spoon, a sterile stirring rod, or a sterile pestle. Alternatively, the antibiotics may be mixed by adding the antibiotics to a sterile container (e.g., a sterile bottle), closing the container (e.g., with a screw top), and shaking the closed container. The topical composition lacking collagenase ointment can be prepared in with a total weight, including all added components, of about 4 g to about 30 g.

### Method of Treatment

The compositions described herein can be administered to a subject in need thereof to treat (e.g., to heal), or to reduce or prevent the development of, wounds or ulcers. The method of treatment includes the following steps.

Diagnostic Step (Optional)

Optionally, the subject may be examined prior to applying a composition of the disclosure to a wound in order to identify an appropriate treatment approach. Prior to diagnosing the wound, a person who is treating the wound washes his/her hands, dries his/her hands with a clean tissue, dresses his/her hands with clean gloves, and examines the wound, the surrounding skin, condition of the wound or ulcer, and the type of the wound/ulcer. Measurements may be taken, e.g., size, depth, etc., and a proper environment for ulcer/wound care may be further prepared.

Once the wound has been assessed, one of the following protocols (one to five) may be selected for treatment of the subject:

Protocol One may be used for an uncomplicated (i.e., non-infected) diabetic ulcer or wound. Protocol Two may be used for an infected wound or ulcer that exhibits irritation, itchiness, inflammation, pus, purulent drainage, slough, and/or devitalized tissue. Protocol Three may be used if the wound/ulcer is difficult to heal, or in cases where methicillin-resistant Staphylococcus aureus (MRSA) is causing infection. In the latter case, ceftaroline fosamil powder may be used in compositions of the disclosure according to Protocol Three. Protocol Four may be used if an infected wound/ulcer is infected by Pseudomonas aeruginosa, or if a subject is originating from, or is in a nursing home, in which case, his/her infection(s) may be caused by Pseudomonas aeruginosa.

A wound may not require a paste or ointment found in the above Compositions section if granulating, uneven, type III collagen is converting to smooth, type I collagen. In this case, an antibiotic(s) may be applied to the wound directly and dressed according to Protocol Five.

If a subject is suffering from systemic lupus erythematosus (SLE) and the wound(s)/ulcer(s) is not infected, then Protocol One may be followed using collagenase ointment and bacitracin zinc-polymyxin B sulfate powder to formulate the paste that impregnates the gauze. If the ulcer(s)/wound(s) is infected, however, then any of the above protocols may be used, accordingly.

Protocol Two may be used if a subject is suffering from an infected surgical wound. If the wound is difficult to heal, then Protocol Three or Protocol Four may be used depending on the infection.

If a subject is suffering from a wound/ulcer caused by gas gangrene, then Protocol One may be followed if the wound/ulcer is not infected. If the wound is infected, then Protocol Two may be used, accordingly. If the wound/ulcer is difficult to heal using Protocol One or Protocol Two, then Protocol Three or Protocol Four may be used depending on the infection.

If the wound is a pressure ulcer or a wound located at the sacral area, or around the hip area, abdomen, lower extremities, shoulder scapula, arms, etc., the treatment selected may also depend on whether the pressure ulcer or wound has pus or purulent drainage. The treatment may also depend on the type of infection(s) and the agents causing it. If the subject is bedridden, the subject may need to adjust himself/herself to avoid or reduce pressure on the wound/ulcer during the healing process, and/or the head of bead (HOB) elevation or the semirecumbent positioning may need to be adjusted. Furthermore, the subject may use support pillows to make themselves more comfortable.

Protocol One

Optionally, a person who is treating the wound (e.g., the subject or a caregiver) washes his/her hands, dries his/her hands with a clean tissue, and dresses his/her hands with clean gloves. Optionally, prior to application of the composition, the wound/ulcer can be cleansed with an appropriate wound cleanser (e.g., 0.05-0.10% normal saline (NS); e.g., Cat. No. 37-6250, Shop Wound Care, Brookfield, Conn.), and can be patted dry with a sterile wound pad or an appropriate, sterile, absorbent tissue (e.g., sterile gauze pad; e.g., Cat. No. 3381, Shop Wound Care, Brookfield, Conn.). One of the mixtures of antibiotics listed in the Compositions section may be applied to the wound (e.g., by directly applying the mixture of powders to the wound), covering the wound bed and wound edges. For example, a mixture of ciprofloxacin and bacitracin zinc-polymyxin B sulfate may be applied to the wound. For a wound that is almost healed, bacitracin zinc-polymyxin B sulfate may be applied to the wound. Next, a paste or ointment listed in the Compositions section may be applied to a sterile material (e.g., a sterile gauze or pad) and applied to the area to be treated or the composition can be directly applied to an area to be treated and covered by a sterile material (e.g., sterile gauze or pad). For example, the paste/ointment containing bacitracin zinc-polymyxin B sulfate and collagenase ointment or the medicated paste/ointment containing ciprofloxacin, bacitracin zinc-polymyxin B sulfate, and collagenase ointment (see above in Compositions) can be applied to a single layer, double layer, triple layer, or quadruple layer (etc.) of sterile gauze pads. The size of the gauze or gauze pad size can be selected to cover all or a sufficient amount of the area to be treated (e.g., depending on the size and depth of the ulcer/wound). If desired, more than one layer of the same or a different sterile material can be applied over the sterile material covering the ulcer/wound. Also, if desired, the area may be further wrapped with KERLIX™ (e.g., Cat. No. 6720, Shop Wound Care, Brookfield, Conn.), or covered with wound pads, as needed. Finally, the bandaged wound can be taped with non-allergic tape to secure the sterile material or wrappings.

The composition can be reapplied to the area to be treated one or more times per day, every other day, one or more times per week, or one or more times per month, until the area has healed. The ulcer/wound can be carefully assessed upon each dressing change. Improvement in wound condition and progress towards healing may start to be noticed gradually. In the first few days of treatment using the disclosed method, daily dressing is optimally performed.

The collagenase ointment improves or increases debridement of the wound/ulcer, and the mixture of antibiotics (e.g., ciprofloxacin and bacitracin zinc-polymyxin B sulfate powder) treats, prevents, inhibits, or reduces infection of the treated area. Revascularization, granulation, and proliferation will follow in a linear way leading to wound healing.

Protocol Two

Optionally, a person who is treating the wound (e.g., the subject or a caregiver) washes his/her hands, dries his/her hands with a clean tissue, and dresses his/her hands with clean gloves. Optionally, prior to application of the composition, the wound/ulcer can be cleansed with an appropriate wound cleanser (e.g., 0.05-0.10% normal saline (NS); e.g., Cat. No. 37-6250, Shop Wound Care, Brookfield, Conn.), and can be patted dry with a sterile wound pad or an appropriate, sterile, absorbent tissue (e.g., sterile gauze pad; e.g., Cat. No. 3381, Shop Wound Care, Brookfield, Conn.). One of the mixtures of antibiotics listed in the Compositions section may be carefully applied to the wound, covering the wound bed and wound edges. For example, a mixture of ciprofloxacin and bacitracin zinc-polymyxin B sulfate can be applied to the wound (e.g., by directly applying the mixture of powders to the wound). Next, a paste or ointment listed in the Compositions section may be applied to a sterile material (e.g., a sterile gauze or pad) and applied to the area to be treated or the composition can be directly applied to an area to be treated and covered by a sterile material (e.g., sterile gauze or pad). For example, the paste/ointment containing ciprofloxacin, bacitracin zinc-polymyxin B sulfate, and collagenase ointment (see above in Compositions) can be applied to a single layer, double layer, triple layer, or quadruple layer (etc.) of sterile gauze pads. The size of the gauze or gauze pad size can be selected to cover all or a sufficient amount of the area to be treated (e.g., depending on the size and depth of the ulcer/wound). Next, the impregnated gauze may optionally be further covered with the paste/ointment. If desired, more than one layer of the same or a different sterile material can be applied over the sterile material covering the ulcer/wound. Also, if desired, the area may be further wrapped with KERLIX™ or covered with wound pads, as needed. Finally, the bandaged wound can be taped with non-allergic tape to secure the sterile material or wrappings.

The composition can be reapplied to the area to be treated one or more times per day, every other day, one or more times per week, or one or more times per month, until the area has healed. The ulcer/wound can be carefully assessed upon each dressing change. Improvement in wound condition and progress towards healing may start to be noticed gradually. In the first few days of treatment using the disclosed method, daily dressing is optimally performed.

The collagenase ointment improves or increases debridement of the wound/ulcer, and the mixture of antibiotics (e.g., ciprofloxacin and bacitracin zinc-polymyxin B sulfate powder) treats, prevents, inhibits, or reduces infection of the treated area. Revascularization, granulation, and proliferation will follow in a linear way leading to wound healing.

Protocol Three

Optionally, a person who is treating the wound (e.g., the subject or a caregiver) washes his/her hands, dries his/her hands with a clean tissue, and dresses his/her hands with clean gloves. Optionally, prior to application of the composition, the wound/ulcer can be cleansed with an appropriate wound cleanser (e.g., 0.05-0.10% normal saline (NS) and can be patted dry with a sterile wound pad or an appropriate, sterile, absorbent tissue (e.g., sterile gauze pad). One of the mixtures of antibiotics listed in the Compositions section may be carefully applied to the wound (e.g., by directly applying the mixture of powders to the wound), covering the wound bed and wound edges. For example, a mixture of bacitracin zinc-polymyxin B sulfate powder, crushed ciprofloxacin, and methicillin powder may be applied to the wound, covering the wound bed and wound edges. Alternatively, a mixture of ceftaroline fosamil powder, bacitracin zinc-polymyxin B sulfate powder (e.g., Polvos de Sulpha First Aid powder; NDC Code: 53145-304, Menper Distributors Inc.) and crushed ciprofloxacin may be applied to the wound (e.g., by directly applying the mixture of powders to the wound), covering the wound bed and wound edges.

Next, a paste or ointment listed in the Compositions section may be applied to a sterile material (e.g., a sterile gauze or pad) and applied to the area to be treated or the composition can be directly applied to an area to be treated and covered by a sterile material (e.g., sterile gauze or pad). For example, the paste/ointment containing collagenase ointment, ciprofloxacin, and bacitracin zinc-polymyxin B sulfate powder may be applied to a single layer, double layer, triple layer, or quadruple layer (etc.) of sterile gauze pads. The size of the gauze or gauze pad size can be selected to cover all or a sufficient amount of the area to be treated (e.g., depending on the size and depth of the ulcer/wound). If desired, more than one layer of the same or a different sterile material can be applied over the sterile material covering the ulcer/wound. Also, if desired, the area may be further wrapped with KERLIX™ (catalog number 6720, Shop Wound Care, Brookfield, Conn.), or covered with wound pads, as needed. Finally, the bandaged wound can be taped with non-allergic tape to secure the sterile material or wrappings.

The composition can be reapplied to the area to be treated one or more times per day, every other day, one or more times per week, or one or more times per month, until the area has healed. The ulcer/wound can be carefully assessed upon each dressing change. Improvement in wound condition and progress towards healing may start to be noticed gradually. In the first few days of treatment using the disclosed method, daily dressing is optimally performed.

The collagenase ointment improves or increases debridement of the wound/ulcer, and the mixture of antibiotics (e.g., ciprofloxacin, methicillin powder, ceftaroline fosamil powder, and bacitracin zinc-polymyxin B sulfate powder) treats, prevents, inhibits, or reduces infection of the treated area. Revascularization, granulation, and proliferation will follow in a linear way leading to wound healing.

Protocol Four Optionally, a person who is treating the wound (e.g., the subject or a caregiver) washes his/her hands, dries his/her hands with a clean tissue, and dresses his/her hands with clean gloves. Optionally, prior to application of the composition, the wound/ulcer can be cleansed with an appropriate wound cleanser (e.g., 0.05-0.10% normal saline (NS)), and can be patted dry with a sterile wound pad or an appropriate, sterile, absorbent tissue (e.g., sterile gauze pad). One of the mixtures of antibiotics listed in the Compositions section may be carefully applied to the wound, covering the wound bed and wound edges. For example, a mixture of bacitracin zinc-polymyxin B sulfate powder and crushed ciprofloxacin can be applied to the wound (e.g., by directly applying the mixture of powders to the wound). Next, a paste or ointment listed in the Compositions section may be applied to a sterile material (e.g., a sterile gauze or pad) and applied to the area to be treated or the composition can be directly applied to an area to be treated and covered by a sterile material (e.g., sterile gauze or pad). For example, a paste/ointment containing collagenase ointment, bacitracin zinc-polymyxin B sulfate powder, and ceftazidime sodium carbonate powder can be applied to a single layer, double layer, triple layer, or quadruple layer (etc.) of sterile gauze pads. The size of the gauze or gauze pad size can be selected to cover all or a sufficient amount of the area to be treated (e.g., depending on the size and depth of the ulcer/wound). Next, the impregnated gauze may optionally be further covered with the paste/ointment. If desired, more than one layer of the same or a different sterile material can be applied over the sterile material covering the ulcer/wound. Also, if desired, the area may be further wrapped with KERLIX™, or covered with wound pads, as needed. Finally, the bandaged wound can be taped with non-allergic tape to secure the sterile material or wrappings.

The composition can be reapplied to the area to be treated one or more times per day, every other day, one or more times per week, or one or more times per month, until the area has healed. The ulcer/wound can be carefully assessed upon each dressing change. Improvement in wound condition and progress towards healing may start to be noticed gradually. In the first few days of treatment using the disclosed method, daily dressing is optimally performed.

The collagenase ointment improves or increases debridement of the wound/ulcer, and the mixture of antibiotics (e.g., ciprofloxacin, ceftazidime sodium carbonate powder, and bacitracin zinc-polymyxin B sulfate powder) treats, prevents, inhibits, or reduces infection of the treated area. Revascularization, granulation, and proliferation will follow in a linear way leading to wound healing.

Protocol Five

A mixture of antibiotics listed in the Compositions section and/or bacitracin zinc-polymyxin B sulfate is applied to the matured wound. The wound can then be covered by a sterile material (e.g., sterile gauze or pad (catalog number 3381, Shop Wound Care, Brookfield, Conn.)). If desired, the area may be further wrapped with KERLIX™ (catalog number 6720, Shop Wound Care, Brookfield, Conn.), or covered with wound pads, as needed. Finally, the bandaged wound can be taped with non-allergic tape to secure the sterile material or wrappings.

Wound Maturation and Wound Dressing

Wound healing will depend on the type, severity, depth, and location of the wound or ulcer. In some cases, a wound can be dressed one or more times daily (or one or more times weekly, if appropriate), using the protocols listed above. Daily dressing changes can be done until slough, devitalized tissue, debris, pus, or purulent drainage are no longer present in a wound. Even if the wound is cleared of slough, necrotic tissue, pus, drainage, or debris, a daily wound dressing schedule may still continue. The daily wound dressing change schedule may last for one day, two days, three days, four days, five days, six days, seven days, eight days, nine days, or longer.

Once a wound exhibits a reduction or absence of slough, devitalized tissue, debris, pus, or purulent drainage, one dressing change every two days can be performed, using the protocols listed above (e.g., Protocol One, Protocol Two, Protocol Three, or Protocol Four). A dressing change performed once every two days (a “two-day dressing change schedule”) can be performed until granulation is present, vascularization of the wound is evident or progressing, and/or healthy type III collagen (i.e., collagen that is pink and uneven) is present. If a wound is severe, then a two-day dressing change schedule can still be followed even if the aforementioned signs of wound maturation are present. The two-day wound dressing change schedule may last for two days, four days, six days, eight days, 10 days, 12 days, 14 days, 16 days, 18 days, 20 days, 22 days, 24 days, or longer. The wound dressing change schedule may be changed from daily wound dressings to once every two days to promote granulation, vascularization, and healthy formation of connective tissue (e.g., type III and type I collagen). The wound dressing schedule may be changed from daily wound dressings to once every three days to further promote granulation, vascularization, and healthy formation of connective tissue (e.g., type III and type I collagen), if present. Lastly, the wound dressing schedule may be changed from once every two days to further achieve wound maturation, as long as wound healing is or continues to be evident (i.e., healthy granulation and vascularization of the wound bed with healthy formation of type III and type I collagen).

Once a wound exhibits granulation, vascularization, and/or healthy type III, uneven collagen, one dressing change every three days can be performed, using the protocols listed above (e.g., Protocol One, Protocol Two, Protocol Three, or Protocol Four). Alternatively, a dressing change may be performed once every four days (a “four-day dressing change schedule”) using the protocols listed above (e.g., Protocol One, Protocol Two, Protocol Three, or Protocol Four), if appropriate. A dressing change performed once every three days or every four days so long as healthy, uneven, type III collagen is apparent and substantially fills (e.g., by 50% or more, such as 60%, 70%, 80%, 85%, 90%, 95%, 99%, or 100%) the wound bed; and/or the wound bed continues to contract; and/or the wound edges are approximating; and/or granulating collagen is merging with the approximating edges; and/or epithelial cells are spreading over the collagen; and/or a scab is forming over the wound. A three-day wound dressing change schedule may last for three days, six days, nine days, 12 days, 15 days, 18 days, 21 days, 24 days, 27 days, 30 days, or longer, following the end of the two-day dressing change schedule. A four-day dressing change schedule may last for four days, eight days, 12 days, 16 days, 20 days, 24 days, or longer, following the end of the three-day dressing schedule, or until complete healing has occurred.

A wound may not require a paste or ointment found in the above Compositions section if granulating, uneven, type III collagen (e.g., thick, uneven collagen) is converting to smooth, type I collagen (e.g., smooth, even collagen). In this case, an antibiotic(s) may be applied to the wound directly, and dressed according to the following procedure (e.g., Protocol Five): a mixture of antibiotics listed in the Compositions section, or bacitracin zinc-polymyxin B sulfate, is applied to the matured wound (e.g., applied directly as a powder). The wound can then be covered by a sterile material (e.g., sterile gauze or pad). If desired, the area may be further wrapped with KERLIX™, or covered with wound pads, as needed. Finally, the bandaged wound can be taped with non-allergic tape to secure the sterile material or wrappings. A wound may be dressed using the above protocol at any time during the treatment, no matter which dressing schedule is being used. For example, if type III collagen (e.g., thick, uneven collagen) is observed to be converting to smooth, type I collagen (e.g., smooth, even collagen), then the aforementioned amended protocol (e.g., Protocol Five) can be used during the two-day dressing schedule, three-day dressing schedule, or four-day dressing schedule.

The above protocols (Protocol One, Protocol Two, Protocol Three, Protocol Four, or Protocol Five) can also be used once every five days, once every six days, once every seven days, once every two weeks, once every month, or longer (e.g., once a month). The above protocols may be used for one week, two weeks, three weeks, four weeks, one month, two months, three months, four months, five months, six months, seven months, eight months, nine months, ten months, eleven months, twelve months, or longer, e.g., until the wound has formed a scab or completely healed. Once a wound has a fully formed scab, the wound can be left open to air (OTA). Results may vary with subject compliance (e.g., following a regimented wound care, and/or complying with prescribed oral antibiotics) and a healthy diet (e.g., a 1,000-2,000 calorie/day diet), as an elevated blood sugar level may inhibit or slow the healing process.

### EXAMPLES

The following examples are put forth so as to provide those of ordinary skill in the art with a description of how the compositions and methods claimed herein are performed, made, and evaluated, and are intended to be purely exemplary of the invention and are not intended to limit the scope of what the inventor regards as his invention.

### Example 1

A male patient had stage four wounds on multiple areas of his body. The wounds were: 1) a tunneling sacral wound; 2) two open infected wounds to outer mid lateral aspect of the left leg; 3) a wound on the internal aspect of the same leg; 4) a deep, open ulcer to the area of peroneus longus of the right leg; 5) two open wounds with yellow/greenish drainage to both ankles. A foul odor permeated from the sacral wound and from the ankle wounds, as well, which indicated that anaerobes and gram-positive bacteria colonized the wounds. Prior to the following disclosed treatment, the patient was previously hospitalized to clear the infections from the wounds described above. At least one doctor recommended that the patient receive treatment in a nursing home, and possible amputation of the legs at the knee joints.

In this example, when a dressing change was performed, the following procedure was followed unless otherwise stated. Furthermore, the following procedure was applied to each wound: the wound was cleansed with 0.9% normal saline (NS) (Nurse Assist, Inc., Reorder No. 6240). A mixture of crushed ciprofloxacin (2.5×500 mg, Aurobindo Pharma USA Inc., NDC Code: 658620077) and bacitracin zinc-polymyxin B sulfate powder (about 7.6 g, POLYSPORIN® powder: polymyxin B sulfate 10,000 units/gram, bacitracin zinc 500 units/gram; Johnson & Johnson, NDC Code: 58232-4033) was thoroughly sprayed in the wound bed. Bacitracin zinc-polymyxin B sulfate powder (about 7.6 g), crushed ciprofloxacin (2×500 mg), and collagenase ointment (about 8 g, SANTYL® ointment: 250 units/gram; Smith+Nephew, NDC Code: 50484-010-30) were formulated into a paste. The resulting paste was placed on 4″×4″ gauze pads. The paste impregnated 4″×4″ gauze pads were then placed on the wound area in a manner maximizing the reach of the paste to every inch of the wound bed. The wound was covered with a triple layer of dry, sterile 4″×4″ gauze pads. Wound pads were then placed on top of the dry, sterile 4″×4″ gauze pads. The resulting wound dressing was then taped with non-allergic tape.

The patient had this procedure done once daily for each wound for the first week. After the first day, the sacral wound dressing was gently removed, and slough, devitalized tissue, and debris were still present. The green/yellowish drainage from the wound remained, as well. Four dressings later (i.e., four days later), the color of the drainage lightened to become a light yellow.

In the ankle wounds, by the fourth dressing change, the slough, devitalized tissue and drainage were diminished. The drainage was thinner and lighter in color, but still slightly yellow. The remaining wounds all showed improvement upon assessment by the fourth dressing change. For example, revascularization with new healthy capillaries, and granulation started in most of the wound beds. The uneven type III collagen started to fill each individual wound bed. Vascularization and granulation appeared more evident in the sacral and ankle wounds by the end of the fifth dressing change.

The daily dressing change for the sacral and ankle wounds continued for eight days. The daily leg wounds dressing change continued for seven days. After seven days, the slough, devitalized tissue, and debris had cleared from the leg wound beds. Dressing change schedules for the legs were changed to once every two days. Sacral and ankle wound dressing changes were set to once every two days after eight daily dressing changes. The two-day dressing change schedule continued for ten changes (i.e., 20 days) for the sacral, ankle, and leg wounds. At that time, the dressing change schedule was changed to once every three days allowing the vascularization and granulation process to gently and healthily progress. The three-day dressing change continued for seven dressing changes (i.e., 21 days) for legs wounds. The three-day dressing change continued for eight dressing changes (i.e., 24 days) for the ankle wounds. The three-day dressing change continued for nine dressing changes (i.e., 27 days) for the sacral wound.

After 48 days (total treatment time), the wound beds of the legs started the healthy contraction process and the edge approximation of the leg wounds were progressing well. The dressing changes for the leg wounds were then set to once every fourth day. The four-day dressing change continued for three dressing changes (i.e., 12 days). After the third dressing change to the leg wounds on the four-day schedule (60 days of total treatment time), epithelial cells appeared to have healthily scabbed the wound and smoothly merged with the smooth healing edges, forming a continued unification of the wound site with the edges. Tiny, sporadic pinkish moist spots appeared at the leg wounds' beds. At this point, the use of medicated paste was suspended. Bacitracin zinc-polymyxin B sulfate powder and crushed ciprofloxacin were sprayed on each wound bed of the leg. The wound was then covered with a double layer of dry, sterile, 4″×4″ gauze pads, wrapped with KERLIX™ and taped with non-allergic tape. Each leg wound was evaluated once every two days, for four days. The leg wounds had completely matured by the second assessment. The leg wounds were left open to air (OTA) at this time (64 days, in total).

After 52 days of treatment, the dressing change schedule for the ankle wounds was set to once every four days. The four-day dressing change continued for three dressing changes (i.e., 12 days). After the third dressing change to the ankle wounds on the four-day schedule (64 days of total treatment time), the use of medicated paste was suspended. The epithelial cells of the ankle wounds scabbed the wound beds. The wound edges had approximated very well. A few pinkish, moist spots were observed on the areas close to the edges of the ankle wounds. The ankle wounds were individually sprayed with crushed ciprofloxacin and bacitracin zinc-polymyxin B sulfate powder. The wounds were covered with a double layer of 4″×4″ gauze pads, wrapped with KERLIX™, and taped with non-allergic tape. Each of the ankle wounds were assessed once every two days. By the third assessment (i.e., six days), the wounds of the ankles had completely matured. Both ankles were left OTA at this time (70 days, in total).

After 55 days of treatment, the sacral wound bed continued contracting in a healthy manner. Smooth healing edges approximated closer with each dressing assessment. The healthy collagen and granulating tissue of the sacral wound bed filled the wound bed. Thus, the sacral wound dressing change schedule was set to once every fourth day. By the second dressing on the four-day schedule (i.e., 8 days), the type III collagen was converting to type I collagen, i.e., smooth, even collagen in the wound bed. The use of medicated paste was suspended at this time. A mixture of bacitracin zinc-polymyxin B sulfate powder and crushed ciprofloxacin was sprayed in the wound bed. A double layer of dry, sterile 4″×4″ gauze pads was used to cover the antibiotics powder. The resulting dressing was then covered with wound pads and taped with non-allergic tape. The sacral wound was evaluated after four days, and the new wound dressing procedure was repeated (i.e., formulated paste was not used). After four more days, the sacral wound was undressed and evaluated. Only a few sporadic pinkish moist spots were observed within the sacral wound parameters. A mixture of bacitracin zinc-polymyxin B sulfate powder and crushed ciprofloxacin was gently sprayed to the wound area. A dry, sterile double layer of 4″×4″ gauze pads was used to cover the layer of antibiotics covering the surface of the wound. The resulting dressing was taped gently with non-allergic tape. The sacral wound was assessed once every two days. After the second wound evaluation (i.e., four days), the sacral wound had matured, and the wound was left OTA at this time (75 days of total treatment). Although the sacral wound tensile was less strong compared to the adjacent skin, and the skin color was slightly off, the sacral wound was healed.

After approximately 2.5 months, every wound on the patient's skin had completely healed. Checkups were continued, and after a year, none of the treated wounds reopened on the patient's skin.

### Example 2

A 101 year-old female patient was suffering from an infected, tunneling sacral wound for approximately one year. Slough, loose strings of infected, discolored tissue was floating on the top of fluid. The loose strings had formed greenish, yellowish/greyish tones in the complex texture of the wound parameters. The devitalized loose strings of tissues were hanging randomly in different directions. A very strong odor permeated from the wound.

In this example, when a dressing change was performed, the following procedure was followed unless otherwise stated: the tunneling wound bed was thoroughly flushed with 0.9% normal saline (NS) (Nurse Assist, Inc., Reorder No. 6240) repeatedly, then was patted dry with sterile 4″×4″ gauze pads. A mixture of bacitracin zinc-polymyxin B sulfate powder (about 9.5 g, POLYSPORIN® powder: polymyxin B sulfate 10,000 units/gram, bacitracin zinc 500 units/gram; Johnson & Johnson, NDC Code: 58232-4033) and crushed ciprofloxacin (2.5×500 mg, Aurobindo Pharma USA Inc., NDC Code: 658620077) was thoroughly sprayed in the wound bed. A paste containing collagenase ointment (about 8 g, SANTYL® ointment: 250 units/gram; Smith+Nephew, NDC Code: 50484-010-30), bacitracin zinc-polymyxin B sulfate powder (about 7.6 g), and crushed ciprofloxacin (2×500 mg) was formulated. The formulated paste was laid on sterile 4″×4″ gauze pads. The resulting paste impregnated 4″×4″ gauze pads were then used to cover the tunneling parts of the wound and the visible area of the wound bed. The wound dressing was next stabilized with non-allergic tape. The patient was positioned to comfort on her left side with head of bed (HOB) elevated to 45 degrees. A support pillow was positioned behind the patient's back. The daily dressing change was done for 2 weeks.

At this time, the slough, devitalized tissue, and discolored drainage had cleared from the wound. The dressing change schedule was then changed to once every two days. Upon the second dressing change following this schedule (i.e., four days), vascularization with new healthy capillaries and granulating tissue was now visible. The two-day dressing change schedule continued for eleven dressing changes (i.e., 22 days). At this time (38 days of total treatment), continued granulation was observed with healthy type III uneven collagen filling the wound. The dressing change schedule was then changed to once every three days. Five dressings were performed using this three-day schedule (i.e., 15 days) and at this point, the tunneling areas had healthily closed and unified with the visible area of the wound bed. Furthermore, healthy collagen had filled the visible area of wound bed. Epithelial cells were spreading and covering the surface of the collagen which converted to type I collagen. The dressing change schedule was changed to once every fourth day to allow the maturation process to finish in a healthy manner. The use of medicated paste was suspended at this time (53 days of total treatment). A mixture of crushed ciprofloxacin and bacitracin zinc-polymyxin B sulfate powder was sprayed on the surface of the wound. The wound was then covered with a triple layer of dry, sterile 4″×4″ gauze pads. The resulting dressing was then covered with wound pads and the dressing was taped with non-allergic tape. This dressing protocol was applied once every four days, and was done for two dressings (8 days). At this point (61 days of total treatment), healthy epithelial cells had formed a smooth scabbing that fully covered the converted type I collagen filling the wound bed. The healthy scabbing had smoothly merged with the healed approximated wound edges. A few moist, dime-sized, pinkish spots were observed near the wound center. A mixture of crushed ciprofloxacin and bacitracin zinc-polymyxin B sulfate powder was again sprayed to the surface of the wound bed. The wound was covered with a double layer of 4″×4″ gauze pads, then taped with non-allergic tape. The wound was assessed every two days over the next six days. After the third assessment, the wound had fully matured. After six weeks of monitoring, the wound stayed intact.

### Example 3

A 47 year-old, female patient was diagnosed with a stage 4, deep tunneling wound to her sacral area, who concurrently had metastasized cancer. Prior to the disclosed treatment, the patient had foaming gel wound dressings that nurses administered per her physician's order, where the wound dressing was changed once every third day. Slough, devitalized/necrotic tissue, and infected greenish/yellow discolored tissue randomly filled the wound bed. Greenish/dark yellowish, odorous drainage filled the wound bed, and soaked whatever was covering the wound. The wound tunneled deep upwards.

The patient received pain medication as ordered to ease any discomforts or pains associated with the wound dressing change. In this example, when a dressing change was performed, the following procedure was followed unless otherwise stated: the patient was placed to comfort on her left side. The tunneling part of the wound was flushed with 0.9% normal saline (NS) (Nurse Assist, Inc., Reorder No. 6240). The wound bed was flushed thoroughly with 0.9% NS. A mixture of crushed ciprofloxacin (2.5×500 mg, Aurobindo Pharma USA Inc., NDC Code: 658620077) and bacitracin zinc-polymyxin B sulfate powder (about 9.5 g, POLYSPORIN® powder: polymyxin B sulfate 10,000 units/gram, bacitracin zinc 500 units/gram; Johnson & Johnson, NDC Code: 58232-4033) powder mix was sprayed in the wound bed reaching under the tunneling edges. A paste containing collagenase ointment (about 8 g, SANTYL® ointment: 250 units/gram; Smith+Nephew, NDC Code: 50484-010-30), bacitracin zinc-polymyxin B sulfate powder (about 7.6 g), and crushed ciprofloxacin (2×500 mg) was formulated and subsequently placed on sterile 4″×4″ gauze pads (Amazon, ASIN B07NDK8XM3). The resulting medication-impregnated 4″×4″ gauze pads were packed inside the tunneling part of the wound and also were used to cover the open part of the wound. Quadruple layers of sterile 4″×4″ gauze pads were used to cover the medicated gauze layers. The resulting wound dressing was then taped with non-allergic tape. This dressing change procedure was done daily for 10 days.

The patient was laid to comfort on her right side, and the head of bed (HOB) was elevated to 45 degrees. A supporting pillow was placed behind patient's lower back to alleviate pressure to sacral area. By the fourth daily dressing change slough and devitalized discolored tissue were starting to break and clear out of the wound. The greenish, dark/yellow drainage appeared lightening towards yellow and by the fifth daily dressing change, the permeating foul smell had subsided. By the ninth daily dressing change, the wound bed started to appear clean of slough, devitalized tissue, and the return flow of the 0.9% NS flush was clear. One more dressing was applied on the daily dressing change schedule which completed the tenth dressing. Upon the removal of this dressing, vascularization with healthy tiny capillaries and granulation had formed in the wound bed. The dressing schedule was then changed to once every two days. Five dressing changes were done on this two-day schedule (10 days).

At this point (20 days of total care), the wound was assessed: vascularization and granulation with type III, thick, uneven collagen continued to fill the wound bed. The tunneling area appeared to be closing progressively with healthy type III collagen filling the gaps. The wound dressing change schedule was then changed to once every three days to promote granulation, closure of the tunneling spaces, contraction of the wound bed, and the healthy approximation of the wound's edges. By the end of the fifth dressing on the three-day schedule (i.e., 15 days), the healthy uneven type III collagen almost filled the wound bed. The wound bed continued to contract progressively, and the wound edges were approximating closer. The edges were smoothly healing, and the granulating collagen was smoothly merging with the approximating edges. Furthermore, epithelial cells were spreading over the collagen scabbing the wound surface.

At this point (35 days of care), the wound dressing schedule was changed to once every four days to allow the maturation process to complete. By the end of the second wound dressing change on the 4-day schedule (8 days later), the type III, uneven collagen converted to type I, smooth, even collagen that filled the wound bed. The epithelial cells spread uniformly producing a healthy scab covering the wound bed. The wound surface had closed with the approximating edges to form a healthy unification of a maturing wound. The use of medicated paste was suspended at this time. A mixture of crushed ciprofloxacin and bacitracin zinc-polymyxin B sulfate powder was sprayed to the wound. The wound was then covered with a triple layer of sterile 4″×4″ gauze pads, which was then covered by wound pads. The resulting wound dressing was taped with non-allergic tape. The wound was reassessed in two days and was found to be almost fully matured except for four minuscule moist spots towards the lower edge of the wound. A mixture of crushed ciprofloxacin and bacitracin zinc-polymyxin B sulfate powder was sprayed on the top of the surface of the wound. The wound was covered with a double layer of dry, sterile 4″×4″ gauze pads. The resulting dressing was taped with non-allergic tape. Upon the two-day assessment, the wound was fully healed and then left open to air (OTA). After six months, the wound did not open.

### Example 4

A female patient had her right kidney removed and was suffering from an infected surgical wound. Prior to administration of the disclosed treatment, the patient was hospitalized for two months in an attempt to heal and close her wound. The wound was foul smelling with copious amounts of pussy yellow, green drainage oozing from the wound that extended from below the right armpit to a few cm above the patient's right groin. The patient was moved to a nursing home for continued treatment in an attempt to heal the surgical wound and received treatment there for approximately six months. The patient was then discharged with her wound still open. The wound and surrounding tissue were swollen, and the wound was still infected. The drainage had become more copious, more greenish, more foul smelling. The patient's skin over her sternum, breasts, abdomen, and the right side of her groin was bloody red and extremely irritated which was now painful even to the thought of touch. The patient was then prescribed morphine sulfate oral, immediate release pills (MSIR) (10 mg, q 4 hrs, PRN) for pain relief.

The surgical wound was thoroughly cleansed with 0.9% normal saline (NS) (Nurse Assist, Inc., Reorder No. 6240). The wound was patted dry with sterile 4″×4″ gauze pads. Crushed ciprofloxacin (1.5×500 mg, Aurobindo Pharma USA Inc., NDC Code: 658620077) was sprayed on the long opened infected surgical wound. A paste of collagenase ointment (about 6 g SANTYL® ointment: 250 units/gram; Smith+Nephew, NDC Code: 50484-010-30), bacitracin zinc-polymyxin B sulfate powder (about 5.7 g, POLYSPORIN® powder: polymyxin B sulfate 10,000 units/gram, bacitracin zinc 500 units/gram; Johnson & Johnson, NDC Code: 58232-4033), and crushed ciprofloxacin (2×500 mg) was formulated and laid on sterile 4″×4″ gauze pads. The resulting paste-impregnated 4″×4″ gauze pads were used to cover the open surgical wound. The wound dressing was then covered by a triple layer of sterile 4″×4″ gauze pads, which were then covered by surgical wound pads, and then taped with non-allergic tape.

Additionally, the patient received 1 tablet of ciprofloxacin, 500 mg, po, bid×2 weeks. The wound dressing change schedule using the protocol above was set daily for the foreseen period. By the tenth daily dressing change, positive results were evident as the bloody red irritated skin color had become pale pinkish, the swelling to the wound site had subsided, and the greenish/yellowish debris and infected discolored drainage had cleared from the wound bed. At this point, the wound dressing change was set for once every two days was to promote angiogenesis, vascularization, and granulation. On day 14, oral ciprofloxacin was suspended as the bloody red irritating condition of the skin had mostly faded. By day 16, the skin redness and irritation completely disappeared. Five dressing changes were performed on the two-day schedule (10 days). Vascularization was progressing well and the pink colored, granulated, uneven type III collagen gradually filled the wound bed during this period. Healthy, smooth, clean, and almost normal wound edges were starting to close in a healthy fashion. After 20 days of treatment, granulating, uneven type III collagen had converted to type I collagen which filled the wound bed. Healing edges were approximating closer. Epithelial cells were surfacing and forming a healthy scabbing.

At this point, the wound dressing schedule was set for once every three days to promote maturation. The use of medicated paste was suspended at this time. A mixture of crushed ciprofloxacin and bacitracin zinc-polymyxin B sulfate powder was sprayed on the wound surface and covered with a triple layer of sterile 4″×4″ gauze pads. The resulting dressing was covered with wound pads, then was stabilized with non-allergic tape. By the 23^{rd} day of treatment, the scabbing had smoothly covered the type I collagen that had filled the wound bed. The healing edges had almost closed and a 6.5 cm long, 0.2 mm wide opening towards the center of the surgical incision remained. A mixture of crushed ciprofloxacin and bacitracin zinc-polymyxin B sulfate powder was used to fill the opening. A triple layer of sterile 4″×4″ gauze pads was then used to cover the wound. The resulting dressing was then taped with non-allergic tape.

By day 26, the wound edges had fully approximated together. The infected surgical wound had fully matured and was left open to air (OTA). The healed surgical wound was revaluated twice weekly for two weeks, then once weekly for two weeks. The wound did not open.

### Example 5

A male patient was diagnosed with multiple severe gastrointestinal illnesses and was fitted with a gastrostomy tube (GT) for feeding. The skin around the insertion point became inflamed and infected. Different methods of treatment were applied to heal the patient's skin condition and with the condition getting worse, the doctors predicted the patient had six months to live. The excoriated skin around the GT insertion in the abdomen had slough with shallow craters. Grey-greenish films were observed on the edges of the spaces between craters, and sticky, yellowish-green slough was observed on the top of the craters. Putrid odor fumed from the site, and the site was sensitive to touch. Furthermore, pus was coming out from the GT insertion point.

In this example, when a dressing change was performed, the following procedure was followed unless otherwise stated: the infected wound site was cleansed with 0.9% normal saline (NS) (Nurse Assist, Inc., Reorder No. 6240), then was patted dry with sterile 4″×4″ gauze pads. The area of the GT tube insertion was cleansed in a similar manner. A mixture of bacitracin zinc-polymyxin B sulfate powder (about 9.5 g, POLYSPORIN® powder: polymyxin B sulfate 10,000 units/gram, bacitracin zinc 500 units/gram; Johnson & Johnson, NDC Code: 58232-4033), and crushed ciprofloxacin (2.5×500 mg, Aurobindo Pharma USA Inc., NDC Code: 658620077) was thoroughly sprayed over the sloughing, excoriated area of the patient's abdominal skin. A paste containing collagenase ointment (about 8 g, SANTYL® ointment: 250 units/gram; Smith+Nephew, NDC Code: 50484-010-30), bacitracin zinc-polymyxin B sulfate powder (about 7.6 g), and crushed ciprofloxacin (2×500 mg) was formulated and laid on sterile 4″×4″ gauze pads. The resulting paste-impregnated 4″×4″ gauze pads were used to cover the excoriated infected area of the patient's skin from the GT insertion point to the edges of the wound. The wound dressing was then further covered by a triple layer of sterile 4″×4″ gauze, which was then covered by sterile wound pads and secured with non-allergic tape. The dressing was changed daily for 11 days.

By the fifth dressing change (day 5), the grey-greenish film building to the edges of the skin between craters started to peel off. By the sixth dressing change (day 6), the sticky yellow drainage in the shallow wound craters and the pus coming around the GT insertion side were starting to subside. By the end of the eleventh dressing change (day 11), the wound area was cleansed of the grey-green films, the yellow drainage, and pus. The GT insertion site and the wound area appeared very clean and pink in color. The wound dressing schedule was changed to once every two days at this time to promote angiogenesis, revascularization, and granulation.

By day 17 (3 dressing changes following the two-day schedule), vascularization appeared across the shallow craters with healthy capillaries. Granular, uneven, healthy pink collagen was filling in each crater of the wound bed. The skin between craters was repairing and spreading. Six more dressing changes were performed using the two-day wound dressing change schedule. By the ninth wound dressing change on this schedule (day 29), the type III, uneven collagen had almost filled every crater of the wound bed. The edges of each one of these craters had been proceeding very well in the approximation process. The skin between craters was clear of films or discoloration and was spreading towards the granulating collagen that filled the small wound beds existing over the patient's abdomen next to the GT insertion site. The wound dressing change schedule was changed to once every three days to allow the granulation process to proceed to the maturation phase in a normal, healthy manner. By the fourth dressing on this schedule (day 41), the previously excoriated, sloughing, infected wound appeared healthy: type III, uneven collagen was converting into type I, smooth surfaced collagen. Epithelial cells were spreading and covered the wound surface from the GT insertion point to the edges of the wound.

A new GT was inserted in the healing insertion site. The use of medicated paste was suspended at this time. The wound dressing change schedule was then changed to once every four days at this time, and the dressing protocol was modified as follows. A mixture of ciprofloxacin and bacitracin zinc-polymyxin B sulfate powder was sprayed on top of the wound surface, then covered with three layers of sterile 4″×4″ gauze pads. The resulting dressing was covered with sterile wound pads and taped with non-allergic tape. After 12 days (i.e., three dressing changes following the modified protocol), the epithelial cells had covered the whole wound surface forming a healthy scabbing to the wound. The GT was fitting well in a tight healthy insertion port in the patient's abdomen. Tiny two nickel-sized pink moist spots were still residing on the right lateral edge. A mixture of crushed ciprofloxacin and bacitracin zinc-polymyxin B sulfate powder was sprayed to the area, and all over the wound surface. A triple layer of 4″×4″ gauze pads was used to cover the wound site and taped with non-allergic tape. On day 55, the wound had completely matured, and the wound was left open to air (OTA). The wound was further monitored twice per week for two weeks, then once per week for two additional weeks.

After the four week period, the wounds were monitored once every two weeks for two months. The patient's wound did not reopen.

### Example 6

A female patient with severe Type II diabetes, systemic, cutaneous and discoid lupus manifestations, diastolic CHF, poor circulation, HTN, RA, and generalized weakness, had multiple recurrent skin ulcers/wounds related to unknown etiology. The patient's multiple skin wounds ranged from a few centimeters to many inches in diameter, especially on lateral and dorsal aspects of both legs. Five small size wounds averaging 1″ to 2.25″ in diameter were observed on both sides of the patient's neck, with one on the right side of the neck, two on the left side, and two on the back of the neck. Three almost-quarter-sized wounds were observed on the patient's scalp. Every ulcer/wound on the patient appeared infected, with slough and devitalized necrotic tissue found in the wound beds. Sticky and viscous drainages of multiple colors were observed and edges of these wounds had excoriated, crusted at some corners, and hardened at others. Furthermore, the wounds were extremely foul smelling. Further assessment showed strings of greenish/greyish tissue growing in most of the wound beds and upon edges. Some wounds appeared like sea algae with loose roots floating around. The toes of the patient's feet had turned either fully or partially dark charcoal in color, with slough, necrosis, sticky viscous drainage and some gangrenous features found on the top or back of each toe.

Each wound was cleaned thoroughly with 0.9% normal saline (NS) (Nurse Assist, Inc., Reorder No. 6240) starting with the toe wounds. A mixture of ciprofloxacin (0.5×500 mg, Aurobindo Pharma USA Inc., NDC Code: 658620077) and bacitracin zinc-polymyxin B sulfate powder (about 1.9 g, POLYSPORIN® powder: polymyxin B sulfate 10,000 units/gram, bacitracin zinc 500 units/gram; Johnson & Johnson, NDC Code: 58232-4033) was sprayed in each wound bed, and upon edges. A paste of collagenase ointment (about 2 g, SANTYL® ointment: 250 units/gram; Smith+Nephew, NDC Code: 50484-010-30), bacitracin zinc-polymyxin B sulfate (about 1.9 g) and ciprofloxacin powder (0.5×500 mg) was formulated and laid on 2″×2″ gauze pads to individually cover each small wound on the toes. Fitted KERLIX™ was used to wrap the dressing, which was then taped with non-allergic tape.

The three wounds of the left dorsal leg laid in linear ascending order down the sura of the left leg, and averaged 4.5 cm wide×6.25 cm long×1.75-2.25 cm deep. The right leg had four wounds, two on the outer aspect, one on the inner aspect, and the last one was on the mid dorsal aspect. A similar treatment protocol to the protocol above was used for the left leg sura wounds. Each wound was covered with paste impregnated 4″×4″ gauze pads (about 4 g, SANTYL® ointment, 500 mg ciprofloxacin, about 3.8 g bacitracin zinc-polymyxin B sulfate powder). Each wound was then taped temporarily with non-allergic tape. Finally, all of the left leg, dorsal aspect wounds were wrapped together with a sheet of KERLIX™ bandage, then taped with non-allergic tape. The inner aspect, right leg wound, which was about 1.5″×2″ and 2 cm deep, was repeatedly cleansed with 0.9% NS, dried, and then sprayed with a mixture of ciprofloxacin (500 mg) and bacitracin zinc-polymyxin B sulfate powder (about 3.8 g) upon the wound bed and edges. A paste containing collagenase ointment (about 5 g), crushed ciprofloxacin (500 mg), and bacitracin zinc-polymyxin B sulfate powder (about 3.8 g) was formulated and laid on 4″×4″ gauze pads. The resulting medicated gauze pads were then used to cover the wound. A double layer of dry sterile 4″×4″ gauze pads covered the paste impregnated layer and was taped temporarily with non-allergic tape. The same medication treatment, e.g., medicated 4″×4″ gauze pads, were used to cover the right leg outer aspect wounds, and the dressings were taped temporarily with non-allergic tape. In a similar fashion, medication-impregnated 4″×4″ gauze pads covered the right leg, dorsal aspect wound. Two dry, sterile 4″×4″ gauze pads were used to cover this wound as well. Non-allergic tape stabilized the 4″×4″ gauze pads temporarily as well. The four right leg wounds were wrapped with one bigger size KERLIX™ sheet, since they were all in close linear proximity. Dressing was then taped with a non-allergic tape. The neck wounds were individually cleansed with 0.9% NS. Bacitracin zinc-polymyxin B sulfate powder was sprayed in each wound bed. A formulated paste of collagenase ointment, ciprofloxacin and bacitracin zinc-polymyxin B sulfate powder was laid on appropriately sized gauze pads, using either 2″×2″ or 4″×4″ gauze pads. The paste impregnated gauze was used to cover each wound individually. A dry, sterile layer of appropriately sized gauze was used to cover the medication impregnated layer. Then, each wound was taped with non-allergic tape. A similar protocol was followed treating the scalp wounds and the neck wounds.

The dressing change schedule was set for every other day for the toes, neck and head wounds for the coming period of time. Each wound was to be thoroughly assessed upon each dressing change. The leg wound dressing changes were set daily for the foreseen period. Each leg wound was to also be assessed very carefully upon each dressing change. By the end of third dressing, the scalp wound beds were the first to start clearing of signs and symptoms (S/S) of infection. Devitalized tissue, slough, and yellow sticky viscous drainage appeared to have abated the wound beds. Edges had cleared of crusty discolored films of infection. By the end of the fifth dressing, vascularization with new healthy capillaries, and granulation with new tissue started to appear in each scalp wound. The dressing change schedule for the scalp wounds was changed to once every third day to allow the granulation phase to proceed in a healthy manner. By the third dressing on the 3-day schedule, wound beds appeared contracting well. The granulating uneven type III collagen had almost filled each scalp wound bed. Epithelial cells started spreading over the collagen in the process of forming a healthy scab on top of each wound. Edges became smooth, clean, and were approximating well. The dressing change schedule for the scalp wounds were changed to once every 4-days at this time. By the end of the second dressing change on the 4-day schedule, epithelial cells had formed healthy scabs on each scalp wound bed. The scab of each scalp wound had merged with the approximating edges forming a healthy unification indicating the wound had matured. Crushed ciprofloxacin (500 mg) and bacitracin zinc-polymyxin B sulfate powder (about 3.8 g) were sprayed on the surface of each wound. The scalp wounds were left open to air (OTA). Each of the scalp wounds were evaluated every two days×3 times. Then once per week×2. The scalp wounds rested in healing mode for good.

By the end of the fourth two-day dressing change, the neck wounds appeared clean of the devitalized tissue, slough, and drainage. By the fifth dressing change of the neck wounds, angiogenesis had started with healthy, fine capillaries appearing in wounds beds. Healthy tissue started to granulate in each wound bed. The dressing change schedule for the neck wounds was changed to once every 3 days to allow the granulation process to continue successfully. By the end of the fourth dressing change of the neck wounds on the new schedule, the healthy type III, uneven collagen was filling each wound bed. Type III collagen was converting to type I, smooth surfaced collagen to finalize filling the wound bed. Epithelial cells were spreading on top of the collagen, forming a scab to cover each wound surface. The use of medicated paste was suspended. A mixture of ciprofloxacin and bacitracin zinc-polymyxin B sulfate powder was sprayed on to top of each wound. A double layer of 4″×4″ gauze pads was used to cover each wound. The wound dressing was then taped with non-allergic tape. Two dressings were performed on the new schedule. At this time, the neck wounds had healthily scabbed, and the wounds' edges completely approximated. Moist spots were noted on the wounds' surfaces. A mixture of ciprofloxacin and bacitracin zinc-polymyxin B sulfate powder was sprayed on top of each wound. A single layer of 4″×4″ gauze pads was used to cover each wound and then was taped with non-allergic tape. The wounds were to be evaluated every 2 days. Upon the 2-day evaluation, the wounds to the patient's neck appeared to have completely matured. The healthy scabbed surface of the contracted wound bed merged with healed, smooth approximating edges. The merger formed a healthy unification of smoothly clean, healed wound. The neck wounds were left open to air (OTA) at this time.

After one week of daily dressing changes to both leg wounds, the wound beds started to clear of devitalized tissue, slough, and yellow viscous sticky drainage. Very tiny, glimmering, crusty discoloration still resided on the wounds' edges. One more daily dressing was applied to all leg wounds. By the end of this last daily dressing change, all wounds of both legs were clear of any signs of infection. The leg wound dressing change was set to once every three-days. By the end of the second dressing change on the new schedule, vascularization with healthy capillaries started spreading in wound beds. Granulating tissue appeared filling the wound beds. By the end of the fourth dressing change, pink, healthy type III collagen continued to fill the wound beds of the leg wounds. The wound beds were contracting in a healthy manner. Clean, smooth wounds edges were approximating well. The use of medicated paste was suspended at this time. A mixture of ciprofloxacin and bacitracin zinc-polymyxin B sulfate powder was sprayed on top of each wound. A double layer of 4″×4″ gauze pads was used to cover each wound temporarily taped with non-allergic tape. Then each leg wound was wrapped with a large portion of KERLIX™, and then stabilized with non-allergic tape. The leg wounds were to be evaluated once every three days on the new treatment regimen. By the second evaluation, all leg wounds had a healthy scab covering the wound bed. Edges of each wound had fully approximated. The wound surface had merged with the healed approximated edges of each wound, thus, forming a healed wound. A mixture of ciprofloxacin and bacitracin zinc-polymyxin B sulfate powder was sprayed on the surface of each wound. A single layer of 4″×4″ gauze pads was used to cover each wound and was then taped with non-allergic tape. The wounds were to be evaluated individually every 2 days. Upon the second 2-day evaluation, the individual leg wounds had completely matured and were left OTA. Each leg wound was assessed twice per week×1 week. Then once weekly×2 weeks. None of these wounds reopened during this period of observation.

Nine daily dressing changes were performed treating the toes' necrotic, devitalized tissue, slough, viscous sticky, pus, and gangrenous wounds. Devitalized tissue, slough, loosely hanging infected stringy textures, pus, sticky viscous drainage, and gangrenous status all started to significantly clear and gradually disappear. By the end of the ninth toe wound dressing change, all wound beds of the toe wounds appeared clean of any S/S of infection. Vascularization with new, healthy capillaries appeared in the wound beds. New tissue was granulating in each individual wound. The wound dressing changes were set at this time to once every 3 days to allow the healthy granulation process to progress normally. Individual wound bed contraction and edge approximation appeared to be progressing well. Type III, uneven collagen appeared filling each individual wound bed by the end of third dressing on the new schedule. The use of medicated paste was suspended at this time. A mixture of ciprofloxacin and bacitracin zinc-polymyxin B sulfate powder was sprayed in each wound bed and covered with a double layer of 4″×4″ gauze pads. The dressing was then wrapped with appropriately fitted portion of KERLIX™ then stabilized with non-allergic tape. Wounds were to be assessed individually once every two days with the new treatment regimen (i.e., sans medicated paste). The uneven type III collagen converted to type I, smooth collagen approximating the individual wound beds by the end of the third assessment. Treatment with the new regimen continued (i.e., sans medicated paste). Wound assessment continued to be every two days. Individual wound bed contraction and individual wound edge approximation continued in a very healthy, healing manner. By the second assessment, the wound bed contraction had finished, and individual wound edges closed. Each toe wound matured and healed. Blood circulation to feet and digits had resumed to normal flow. The dark charcoal-colored feet and toes appeared to return to the patient's normal skin color. The toe wounds were evaluated twice per week×2 weeks, once/week×2 weeks. The toe wounds did not open again. The toe wounds did not reveal any S/S of irritation or tendency for infection.

### Example 7

A male patient hard large, infected wounds on both legs. The wounds were draining on the floor and emitted a pungent odor. The patient was hospitalized four times prior to being in a nursing home without a resolution to the patient's leg wounds. The doctors at the hospital discussed the strong possibility that the patient's left leg may be amputated at the knee joint. The doctors also discussed amputating the patient's right leg 2 inches above the ankle. The patient had type II diabetes which was not controlled and had cancer that induced venous insufficiency to both of his legs. The left leg had a dorsal open wound about 4″ wide×4.5″ long, and about 1.25″ deep. The right leg dorsal wound was about 2.5-3″ wide and approximately 1″ deep. Pus and thick, yellow/brownish, sticky drainage leaked through and stained the dressing that was covering the wound.

A second wound on the right leg laid was observed on the right ankle. It was in 4.6″ in diameter and about 1-1.25″ deep varying from one point in the wound to another. The right ankle/foot wound drainage was moderate, viscous, sticky, and yellow/brown in color. The sticky viscous drainage had a tinge of bronzy greenish color towards the edges of the wound.

Treatment started with the right ankle wound. The prior dressing was removed. The wound was thoroughly cleansed with 0.9% NS and patted dry with sterile 4″×4″ gauze pads. A mixture of ciprofloxacin powder (500 mg, Aurobindo Pharma USA Inc., NDC Code: 658620077) and bacitracin zinc-polymyxin B sulfate powder (about 1.9 g, POLYSPORIN® powder: polymyxin B sulfate 10,000 units/gram, bacitracin zinc 500 units/gram; Johnson & Johnson, NDC Code: 58232-4033) was thoroughly sprayed in the wound bed. A paste containing collagenase ointment (about 3 g, SANTYL® ointment: 250 units/gram; Smith+Nephew, NDC Code: 50484-010-30), crushed ciprofloxacin (0.5×500 mg), and bacitracin zinc-polymyxin B sulfate powder (about 3.3 g) was formulated and laid on 4″×4″ gauze pads. The paste impregnated 4″×4″ gauze pads were used to cover the wound. A triple layer of sterile 4″×4″ gauze pads was used to cover the paste impregnated layer, which was then wrapped with KERLIX™ The wound dressing was then taped with a non-allergic tape. A loose clean sock was put on the patient's foot. The patient's right foot was placed on a comfortable footstool.

The left leg dressing was removed for assessment and dressing change. The wound was still draining, but the dressing held in place. The wound's odor was still permeating in the immediate area around the patient. The wound's dressing was unwrapped and discarded properly. The skin area around the wound was cleansed with soap and water and was gently patted dry. The wound was thoroughly cleansed with 0.9% NS and gently dried with sterile 4″×4″ gauze pads. A mixture of crushed ciprofloxacin powder (0.5×500 mg) and bacitracin zinc-polymyxin B sulfate powder (about 2.4 g) was sprayed in the wound bed. A paste containing collagenase ointment (about 3 g), crushed ciprofloxacin powder (500 mg) and bacitracin zinc-polymyxin B sulfate powder (about 3.3 g) was formulated and then laid on 4″×4″ gauze pads. The paste impregnated 4″×4″ gauze pads were used to cover the wound. A triple layer of dry, sterile 4″×4″ gauze pads was used to cover the medication impregnated layer. The wound was wrapped with KERLIX™ then taped with non-allergic tape.

Daily dressings following the above protocol continued. Each wound was to be carefully evaluated and thoroughly assessed upon each dressing change. Daily dressings continued for eight consecutive days for the right ankle wound. By the end of the eighth wound dressing change, the wound bed and wound edges on the right ankle appeared clean of devitalized tissue, slough, viscous sticky multi-colored drainage and any other signs of infection. Vascularization with new healthy capillaries, and granulating new tissue was starting to appear in the wound bed. The wound dressing change schedule for the right ankle was set to once every two days to allow the granulation process to proceed successfully. The same medication treatment regimen, and wound wrapping procedures continued in the process of the wound care for the right ankle. By the end of the sixth dressing change on the two-day schedule, the vascularization and granulation of the new tissue appeared to be progressing in a healthy manner, demonstrated by the type III, pink, strong collagen that had approximated the wound bed. The wound dressing change schedule for the right ankle was changed to once every 3 days. By the end of the first dressing change on the new schedule, uneven type III collagen was converting to smooth, healthy type I collagen rising and spreading in the wound. Epithelial cells were spreading to cover the collagen surface. The wound bed was contracting, and edges continued to approximate progressively to close the wound in a healthy manner. The use of medicated paste was suspended at this time. A mixture of ciprofloxacin powder and bacitracin zinc-polymyxin B sulfate powder was sprayed on top of wound surface including edges. A triple layer of 4″×4″ gauze pads was used to cover the wound. The right ankle wound was to be assessed every three days. By the end of the second assessment, epithelization process had completed, forming a smooth, healthy scabbing that covered the smooth type I collagen of the wound bed. The right ankle wound edges had fully approximated. The wound had closed and matured. The wound was left open to air (OTA) at this time. The wound was observed carefully and assessed twice per week×1 week, then once weekly×2 weeks. The right ankle wound did not open again during this period of time. Nor did it show any signs of irritation, or tendencies to get infected again.

The daily dressing change continued for the wound of the dorsal aspect of the left leg for seven days. By end of the seventh daily dressing change, the wound of the dorsal aspect of the left leg appeared to have cleared of devitalized tissue, slough, and multicolored viscous sticky in the wound bed and edges. The foul smell had subsided as well. Vascularization with new healthy capillaries, and granulation of new healthy tissue had started in the wound bed. The dressing change schedule for the wound of the dorsal aspect of the left leg was changed to once every 2 days with the same medication treatment regimen (e.g., antibiotic spray with collagenase/antibiotic paste), and clinical wound wrapping procedure. The left leg wound was carefully assessed upon each new dressing change. It was evident that healthy vascularization, and healthy granulation continued towards the projected healing goal. Pink, strong, uneven type III collagen was spreading and rising in the wound bed. By the end of the fifth dressing change of the wound of the dorsal aspect of the left leg, the uneven, healthy, pink type III collagen had filled the wound bed. The type III collagen was converting into type I, smooth surfaced collagen. Epithelial cells were starting to cover the wound surface. The wound bed was contracting, and smooth, clean wound edges were approximating properly towards a wound closure. The wound dressing schedule was changed to once every 3 days. The use of medicated paste was suspended. The wound wrapping procedures were to continue in this manner (i.e., without collagenase/antibiotic paste) with the new schedule. By the end of the first dressing change on the new schedule, epithelial cells had fully spread in the wound bed, forming a healthy scab. The healthy scab had merged with the smooth, clean, approximating edges, forming a healthy unification. Three pink, slightly moist, penny sized dots appeared towards the upper edge of the wound surface. A mixture of ciprofloxacin powder and bacitracin zinc-polymyxin B sulfate powder was sprayed on the wound surface. A double layer of dry, sterile 4″×4″ gauze pads was used to cover the wound bed and was wrapped gently with KERLIX™, then taped with non-allergic tape. The wound was to be assessed every two days. By the end of the second wound assessment, the pink moist dots had evanesced. The wound to the dorsal aspect of the left leg had matured. The color to the healed wound area was still off from the patient's normal skin color. The wound tensile strength was less than the adjacent skin. Nevertheless, the patient's wound of the dorsal aspect of the left leg had fully healed. The healed wound condition was evaluated twice per week×1 week, then once per week×2 weeks. The wound of the dorsal aspect of the left leg did not open again during this period. The wound did not show any irritations, nor any S/S of being infected again. The patient received scheduled physical therapy at home and started to regain strength to the left lower extremity as well. The patient was able to stand and walk utilizing a cane two and a half weeks after starting the physical therapy. Neither the left leg wound, nor the right ankle wound opened again for the following three years after they healed.

### Example 8

A female patient with type II diabetes, COPD, CHF, GERD, recurrent renal calculi, osteo-arthritis, obesity and generalized weakness, had recurrent ulcers mostly to her lower extremities. The patient lived by herself and weighed 360-375 lbs. Both of her legs had wounds: the left posterior, and right leg, mid outer lateral wounds were oozing thick greenish/yellowish, odorous discharge. The wound of the left leg started dorsally below the popliteal fossa and twisted laterally to the outer aspect on the patient's left leg sura. The wound was about 10.5 cm long and about 3 cm deep. Devitalized tissue, slough, and randomly split pieces of devitalized tissue were present. Viscous, brownish pus filled deep craters burrowed in the wound bed.

The wound of the left foot laid from the top middle of the left foot extending towards the inner aspect of the left foot ankle. The wound had an oblong shape. The wound ranged from 8.5 cm at the widest to 2 cm at the narrow ends. The wound had signs and symptoms (S/S) of infection similar to the wound of the dorsal aspect of the left leg.

Two wounds laid on the mid outer lateral aspect of the right leg, and on the top of the left foot. The wound on the mid outer aspect of the right leg appeared deeper in the center and more shallow towards the edges. The wound had an irregular circular shape. Its diameter in the middle was 8.32 cm and was about 3.3 cm deep in the center. The wound averaged about 2 cm deep towards the edges.

The wound of the right foot was like a big circle with very irregular circumference, and fissured edges. There were elevations and dents within the wound bed. Some of the elevated spots appeared inflamed red with pus oozing from them. The dented areas appeared darkish with viscous multi-colored drainage. The diameter of the wound of the right foot was approximately 7-7.5 cm.

The patient was then told to avoid concentrated sugars since elevated blood glucose levels can impede wound healing. The patient's PCP placed the patient on a 2000 calorie diabetic diet and added Farxiga (100 mg, po, daily) to patient's medication regimen. The wound dressing change schedule was set daily for the foreseen period. Each wound was to be thoroughly assessed upon each dressing change.

On the next day, the heel of the left foot was placed on an elevated foot-stool. The wound was thoroughly flushed with 0.9% NS. The wound bed was patted dry with sterile 4″×4″ gauze pads. A mixture of bacitracin zinc-polymyxin B sulfate (about 2.4 g, POLYSPORIN® powder: polymyxin B sulfate 10,000 units/gram, bacitracin zinc 500 units/gram; Johnson & Johnson, NDC Code: 58232-4033) and crushed ciprofloxacin (1.5×500 mg, Aurobindo Pharma USA Inc., NDC Code: 658620077) was sprayed thoroughly in the wound bed. A paste of collagenase ointment (about 3 g, SANTYL® ointment: 250 units/gram; Smith+Nephew, NDC Code: 50484-010-30), crushed ciprofloxacin (500 mg), and bacitracin zinc-polymyxin B sulfate powder (about 2.4 g) was formulated and placed on 4″×4″ gauze pads. The paste impregnated gauze pads were used to cover the wound bed. A triple layer of 4″×4″ gauze pads was used to cover the medication impregnated layer, which was then wrapped with KERLIX™ and taped with non-allergic tape.

The same medication treatment regimen and wrapping procedure was followed to treat the wound of the right foot. The wound bed was flushed with 0.9% NS and dried with sterile 4″×4″ gauze pads. A mixture of crushed ciprofloxacin (1.5×500 mg) and bacitracin zinc-polymyxin B sulfate powder (about 3.3 g) was sprayed in the wound. A paste containing collagenase ointment (about 5 g), crushed ciprofloxacin (500 mg), and bacitracin zinc-polymyxin B sulfate powder (about 4.8 g) was formulated and laid on sterile 4″×4″ gauze pads. The paste impregnated 4″×4″ gauze pads were used to cover the outer lateral wound of the right leg. A triple layer of sterile 4″×4″ gauze pads was used to cover the medication impregnated layer, which was then wrapped with KERLIX™ and taped with non-allergic tape.

By the end of the eighth daily dressing change, all the wounds, except the mid outer lateral wound of the right leg, appeared clean. Vascularization with new, healthy blood vessels was observed, and new red connective tissue appeared granulating in each wound bed. The wound dressing change schedule was changed to once every 2 days for both wounds of the feet, and the dorsal aspect wound of the left leg. This was to allow the granulation process to proceed in a healthy manner. The wound of the mid outer lateral aspect of the right leg was dressed daily using the above protocol for the time being. The wound medication treatment regimen, and clinical wound wrapping procedure continued for each of the other three wounds on the new 2-day wound dressing schedule. Vascularization and granulation appeared to be progressing upon assessment with each new wound dressing change. By the end of the fifth dressing change on the 2-day schedule, the granulating, uneven type III collagen appeared to be filling the wound beds and starting to convert into type I, smooth surfaced collagen. The wound beds were contracting progressively, and wound edges were approximating. Epithelial cells from wound beds and edges were spreading to cover the surfaces of the wounds. The wound dressing schedule was changed to once every three days at this time to allow the granulation phase to proceed successfully into the maturation phase. By the end of the first dressing on the 3-day schedule, the use of medicated paste was suspended. The wound treatment continued as follows: a mixture of crushed ciprofloxacin and bacitracin zinc-polymyxin B sulfate powder was sprayed upon each wound. Each wound was covered with a double layer of 4″×4″ gauze pads, then was wrapped with KERLIX™ and taped with non-allergic tape. The wounds were to be assessed every three days. By the end of the second assessment, the wounds of the feet had fully formed healthy scabs. The wound edges had approximated. Each wound of each foot had fully closed and completely matured and were left open to air (OTA) at this time. By the end of the second assessment on the 3-day schedule of only antibiotic treatment (e.g., treatment sans medicated paste), the wound of the dorsal aspect of the left leg still had four moist, pink spots in different areas of the wound surface. A mixture of crushed ciprofloxacin and bacitracin zinc-polymyxin B sulfate powder was sprayed upon the wound surface and covered with a double layer of 4″×4″ gauze pads. The dressing was then wrapped with KERLIX™ and then taped with non-allergic tape. The wound was to be evaluated in two days. Upon the 2-day assessment, the wound surface had smoothly scabbed with healthy shining epithelial cells and the wound edges had closed, forming a seamless line. The wound of the dorsal aspect of the left leg was left OTA at this time.

Ten more dressings on the daily dressing change schedule were performed on the right leg, mid outer lateral aspect wound using the medicated paste protocol. By the end of the 10^{th} daily dressing change for this wound, the wound bed and wound edges appeared very clean. Devitalized tissue, slough, pus, viscous sticky drainage, etc. had cleared out of the wound bed. Edges appeared to be smooth and clean. Vascularization with new, healthy blood vessels was observed in the wound bed. New, healthy red connective tissue was granulating in the wound bed as well. The wound dressing schedule was changed to once every 2 days to allow the granulation process to proceed. By the end of the 6^{th} dressing change, the healthy, pink, uneven type III collagen appeared to be approximating the wound bed. Type III, healthy, uneven collagen was converting into the smooth surfaced type I collagen. The wound bed was progressively contracting, and the wound edges were approximating. Epithelial cells were spreading on the wound surface to form a healthy scab on the surface of the wound. The wound dressing schedule was changed to once every 3 days to allow the granulation process to complete into maturation. The use of medicated paste was suspended at this time. The following protocol was used: a mixture of crushed ciprofloxacin and bacitracin zinc-polymyxin B sulfate powder was thoroughly sprayed upon the wound bed and edges. A double layer of sterile 4″×4″ gauze pads was used to cover the wound which was then wrapped with KERLIX™ and taped with non-allergic tape. The wound was to be evaluated every two days. By the 3^{rd} evaluation, the mid outer lateral wound of the right leg had fully contracted. Epithelial cells had formed a complete, shiny scab covering the wound bed. The edges had approximated, maturing into a seamless, healed wound. Each of the four wounds were evaluated twice per week×2 weeks, then once per week×3 weeks. None of these wounds reopened during this period of time nor had shown any S/S of infection. Furthermore, none of the wounds showed any tendencies to open again in the two and half months of care.

### Example 9

A male patient in his mid-forties with type II diabetes, hypertension, gout, asthma, acid reflux, and amputated toes on the right foot due to gangrene, had an infected wound in the amputated area. Furthermore, the patient had another open wound around the great toe of the left foot. The patient was not ambulating anymore due to these infections. The patient laid in bed most of the time and had a sacral wound. The sacral wound bed was wide open and was tunneling to the sides and towards the upper end. Devitalized tissue, slough, and sticky viscous purulent drainage had settled in the deep sacral wound due to immobilization. The patient was instructed to perform blood glucose home assessments at least twice per day, to follow a 2,000 calorie diabetic diet prescribed by a physician, to follow the physician's prescribed medication regimen, and to comply with physician's follow up-appointments, including A1C tests. The PCP further prescribed Levaquin (750 mg, po, daily×5 days with 1 refill), and Metformin (1000 mg, twice per day).

The wounds were dressed daily for the foreseen period. Each wound was to be individually evaluated upon each dressing change. The sacral wound was repeatedly flushed with 0.9% normal saline (NS) (Nurse Assist, Inc., Reorder No. 6240) until the return flow was lighter in color. The wound was patted dry with sterile gauze 4″×4″ gauze pads. A mixture of bacitracin zinc-polymyxin B sulfate (about 3.8 g, POLYSPORIN® powder: polymyxin B sulfate 10,000 units/gram, bacitracin zinc 500 units/gram; Johnson & Johnson, NDC Code: 58232-4033) and crushed ciprofloxacin (2.5×500 mg, Aurobindo Pharma USA Inc., NDC Code: 658620077) was sprayed thoroughly in the wound bed and tunneling areas. A paste containing collagenase ointment (about 5 g, SANTYL® ointment: 250 units/gram; Smith+Nephew, NDC Code: 50484-010-30), bacitracin zinc-polymyxin B sulfate (about 2.9 g), and crushed ciprofloxacin (500 mg) was formulated and placed on 4″×4″ gauze pads. Some of the paste impregnated 4″×4″ gauze pads were embedded within the tunneling areas of the sacral wound. The remaining paste impregnated 4″×4″ gauze pads were used to cover the open wound bed. A triple layer of a dry sterile 4″×4″ gauze pads was used to cover the paste impregnated layer, which was covered by wound pads and taped with non-allergic tape.

The wound of the right foot was carefully cleansed with 0.9% NS and patted dry with sterile 4″×4″ gauze pads. A mixture of bacitracin zinc-polymyxin B sulfate (about 2.4 g) and crushed ciprofloxacin (500 mg) was sprayed to the wound bed. A paste containing collagenase ointment (about 3 g), bacitracin zinc-polymyxin B sulfate powder (about 2.4 g), and crushed ciprofloxacin (500 mg) was formulated and placed on 4″×4″ gauze pads. The paste impregnated 4″×4″ gauze pads were used to cover the wound bed. A double layer of dry, sterile 4″×4″ gauze pads was used to cover the paste impregnated 4″×4″ gauze pads, which was then wrapped with KERLIX™. The wound dressing was then wrapped with non-allergic tape.

Upon further assessment, the left toe was somewhat swollen and greyish in color. The wound bed of the great toe of the left foot appeared tan/yellowish. The wound bed was repeatedly cleansed with 0.9% NS and patted dry with sterile 4″×4″ gauze pads. A mixture of bacitracin zinc-polymyxin B sulfate (about 2.9 g) and crushed ciprofloxacin (500 mg) was sprayed in the wound bed. A paste of collagenase ointment (about 3 g), bacitracin zinc-polymyxin B sulfate (about 2.4 g), and crushed ciprofloxacin (500 mg) was formulated and laid on 4″×4″ gauze pads. The paste impregnated 4″×4″ gauze pads were used to cover the wound bed. A double layer of sterile 4″×4″ gauze pads was used to cover the paste impregnated layer, which was then wrapped with KERLIX™ and taped with non-allergic tape. Eight daily dressing changes were performed to the wound of the right foot. Devitalized tissue, slough, viscous drainage, and other contaminants were cleared out of the wound with each dressing change. By the end of the eighth daily dressing change, the wound of the right foot appeared clean, and angiogenesis started with new, healthy blood vessels proliferating in the wound bed. New, healthy, red colored tissue granulated the wound bed as well. The dressing schedule for the wound of the right foot was changed to once every 2 days at this time to promote the granulation process. By the end of the fifth dressing change on the new 2-day schedule, the wound bed was contracting. The uneven type III pink, strong collagen was filling the wound bed. Type III, uneven collagen started to convert to type I, smooth collagen. Epithelial cells were spreading from the wound bed and edges, forming a surface on the collagen layer. The wound dressing schedule was changed to once every 3 days at this time. By the end of the second dressing on the new 3-day schedule, the wound surface appeared uniformly smooth with epithelial cells starting to scab. A few pink, moist spots remained in the wound. The use of medicated paste was suspended at this time. A mixture of ciprofloxacin and bacitracin zinc-polymyxin B sulfate powder was sprayed on the wound surface. A double layer of clean 4″×4″ gauze pads was used to cover the wound, which was then wrapped with KERLIX™ and taped with non-allergic tape. The wound was to be evaluated every 2 days using the ciprofloxacin and bacitracin zinc-polymyxin B sulfate treatment. By the end of the third evaluation, the wound of the right foot appeared to be completely matured. A healthy epithelial surface was spread on top of the wound of the right foot, which was left open at this time.

Nine daily wound dressings using the collagenase/antibiotic paste were performed on the wound of the great toe of the left foot. Similar to the wound of the right foot, with each dressing change assessment the wound showed improvement. Slough, devitalized tissue, viscous sticky drainage appeared loosening and clearing out of the wound bed. By the end of the ninth daily dressing change, the wound of the great toe of the left foot appeared clean. Vascularization started with new, healthy blood vessels proliferating in the wound bed. The dressing change schedule was changed to once every 2-days at this time. By the end of the fifth dressing change, the pink colored type III, uneven collagen was filling the wound bed and was starting to convert into smooth surface type I collagen. Contraction of the wound was also observed. Epithelial cells were spreading from the edges and wound bed to cover the collagen layer. The wound dressing change schedule was changed to once every 3 days. By the end of the first dressing change on this schedule, the collagen had smoothened to type I collagen. Epithelial cells continued to scab the wound surface. The use of medicated paste was suspended at this time. A mixture of ciprofloxacin powder and bacitracin zinc-polymyxin B sulfate powder was sprayed in the wound. A double layer of 4″×4″ gauze pads was used to cover the wound which was wrapped gently with KERLIX™ and taped with non-allergic tape. The wound was to be assessed every 2 days using the antibiotic treatment (i.e., sans collagenase/antibiotic paste). By the end of the second dressing, the wound bed had fully contracted, and the wound was left open to air (OTA) upon this time.

By the end of the tenth daily dressing change following the collagenase/antibiotic paste protocol, the sacral wound tunneling areas, and the open wound bed area appeared clean. New, healthy blood vessels were proliferating through the entire wound area. New, red colored, healthy tissue was granulating through the bed as well. The wound bed was starting to contract. The sacral wound edges were smooth and clean. The dressing change schedule of the sacral wound was changed to once every 2 days at this time using the same medicated treatment and clinical dressing procedures. By the end of the end of the 6^{th} dressing change, tunneling areas were starting to close in a healthy manner. Pink colored type III collagen was filling the tunneling areas and the wound bed. The wound dressing change schedule was changed to once every 3 days. By the end of the second dressing on the new schedule, collagen was converting into smooth layered type I collagen. Tunneling areas were closing and epithelial cells started scabbing the surface of the wound. The use of medicated paste was suspended at this time and the following protocol was followed: a mixture of crushed ciprofloxacin and bacitracin zinc-polymyxin B sulfate powder was sprayed on the wound surface. A double layer of 4″×4″ gauze pads was used to cover the wound bed, which was covered by wound pads and taped with non-allergic tape. The sacral wound was to be evaluated once every 2 days using the new wound dressing protocol. By the end of the second evaluation, the wound was contracting in a heathy manner. The smooth, clean edges of the wound were approximating. Four pink, nickel sized-moist spots had spread in the wound surface. The wound was redressed using the modified protocol and was to be evaluated every 2 days. By the 2^{nd} evaluation, epithelial cells had formed a smooth surface covering the wound. The smooth scab had merged with the approximating edges to form a continued unification of a healthy matured wound. The wound was left OTA at this time.

The wounds were monitored and assessed individually twice per week×2 weeks. Then once per week×3 weeks. None of these wounds reopened during this period of time, nor did they show any S/S of infection or S/S of irritation.

### Example 10

A 58 year-old male patient with blood glucose levels greater than 200 mg/dl and an amputated left foot had an infected surgical wound where the amputation was done (the amputation took place two months prior to treatment of the wound). The amputation dressing was colored dark, brown, yellowish. The great and second toes of the right foot had been recently amputated and were also infected.

The left foot amputation site had formed a wet zone within and around the whole amputation area. The amputation bed was filled with devitalized strings of tissue and sticky, purulent drainage. The wound bed and edges were multilayered with multi-colors mapping the amputation site. The recent amputation site of the right foot great and second toes had viscous, light yellowish drainage with itchiness/pain reported by the patient. The patient also suffered from a sacral wound in which the areas of adjacent skin appeared reddish in color. The sacral wound bed had devitalized tissue, puffy slough, and viscous, purulent drainage. Strings of loose slough with permeating foul odor were entangled among the mesh. The sacral wound had tunneled upwards and laterally. In addition to the necessary supplies for wound treatment, the PCP ordered Clindamycin (300 mg, po, q 6 hrs.×1 week).

The wound dressing was started daily using the procedure that follows. The wound of the right foot, where the great and second toes were, was thoroughly cleansed with 0.9% normal saline (NS) (Nurse Assist, Inc., Reorder No. 6240). A mixture of crushed ciprofloxacin (500 mg, Aurobindo Pharma USA Inc., NDC Code: 658620077) and bacitracin zinc-polymyxin B sulfate powder (about 2.9 g, POLYSPORIN® powder: polymyxin B sulfate 10,000 units/gram, bacitracin zinc 500 units/gram; Johnson & Johnson, NDC Code: 58232-4033) was thoroughly sprayed in the wound bed. A paste containing collagenase ointment (about 3.5 g, SANTYL® ointment: 250 units/gram; Smith+Nephew, NDC Code: 50484-010-30), crushed ciprofloxacin (500 mg), and bacitracin zinc-polymyxin B sulfate powder (about 2.9 g) was formulated and laid on sterile 4″×4″ gauze pads. The paste impregnated 4″×4″ gauze pads were used to cover the wound of the right foot. A triple layer of sterile 4″×4″ gauze pads was used to cover the paste impregnated layer, which was then wrapped with KERLIX™ and taped with non-allergic tape. The wound of the left foot was then dressed using the same protocol that was followed in the dressing of the wound of the right foot.

The sacral wound bed was thoroughly flushed with 0.9% NS and patted dry with dry sterile 4″×4″ gauze pads. A mixture of crushed ciprofloxacin (3×500 mg) and bacitracin zinc-polymyxin B sulfate powder (about 6.2 g) was thoroughly sprayed in the wound bed. A paste containing collagenase ointment (about 8 g), crushed ciprofloxacin (2.5×500 mg), and bacitracin zinc-polymyxin B sulfate powder (about 5.2 g) was formulated and laid on 4″×4″ gauze pads. The paste impregnated 4″×4″ gauze pads were used to cover the wound bed. A triple layer of dry, sterile 4″×4″ gauze pads was used to cover the paste impregnated layer, which was then covered with wound pads and taped with non-allergic tape.

Nine daily dressings were performed on the wound of the right foot of the recently amputated great and second toes. The wound of the right foot demonstrated improvement with each dressing change as devitalized tissue, slough, and other contaminants were gradually clearing out of the wound bed. By the end of the 10^{th} dressing change, the right wound bed and edges appeared clean. Vascularization had started with new blood vessels proliferating in the wound bed. New connective tissue was now growing in the wound bed. The dressing change schedule for the wound of the right foot was changed to once every 2 days at this time. By the end of the 7^{th} dressing change on the new 2-day schedule, the wound bed appeared to be contracting and the wound edges appeared smoother and cleaner. Type III collagen almost filled the wound bed and was starting to convert to smooth type I collagen. Epithelial cells were spreading from the wound center and wound edges, forming a scab. The dressing change schedule for the wound of the right foot was changed to once every 3 days to allow the granulation process to continue successfully and to proceed to the full wound maturation phase. The use of medicated paste was suspended at this time and the protocol was amended as follows. A mixture of crushed ciprofloxacin and bacitracin zinc-polymyxin B sulfate powder was sprayed on the surface of the wound of the right foot. A double layer of sterile 4″×4″ gauze pads covered the wound, and was wrapped with KERLIX™. The wound dressing was then taped with non-allergic tape. The wound was to be evaluated every 2 days using the amended protocol. By the third evaluation, the epithelial cells had covered the type I, smooth collagen layer that had completely filled the wound bed. The wound bed had fully contracted, and the scab had fully merged with the healed edges, forming a clean unification of a fully healed wound. The wound of the right foot was left open to air (OTA) at this time.

Analogous to the right foot, nine daily dressings were performed on the patient's wound of the left foot, using the same 0.9% NS cleansing regimen, similar medication regimen (e.g., spraying with antibiotics then covering with collagenase ointment/antibiotic paste impregnated gauze pads), and similar clinical wound wrapping procedures. By the end of the 9^{th} daily dressing change to the wound of the left foot, the wound appeared clean. Vascularization and healthy connective tissue were proliferating in the wound bed. The edges were smooth and clean. The wound dressing change schedule was set at this time for once every 2 days was to allow the granulation process to proceed successfully. By the end of the 6th dressing change on this schedule, the wound bed appeared contracting progressively. Type III collagen appeared to be filling the wound bed. The wound edges continued healing and approximating. The wound dressing change schedule was changed to once every 3 days at this time. By the end of the 2^{nd} dressing, type III collagen had converted into type I collagen. Epithelial cells originating from the wound bed and edges were covering the collagen surface and forming a healthy scab. The use of medicated paste was suspended at this time and the wound dressing protocol was amended as follows. A mixture of crushed ciprofloxacin and bacitracin zinc-polymyxin B sulfate powder was sprayed in the wound. A double layer of 4″×4″ gauze pads was used to cover the wound bed, which was wrapped gently with KERLIX™ and taped with non-allergic tape. The wound was to be evaluated every two days on the new dressing protocol (i.e., only antibiotics treatment). By the 2^{nd} evaluation, epithelial cells had formed a complete, healthy scab which had merged with the healed edges. The wound of the left foot had fully matured and was left OTA at this time.

Similar to the wounds of the feet, 10 daily dressing changes were performed on the sacral wound using the same daily dressing protocol of washing the wound with 0.9% NS, medicating the wound, and dressing the wound with medicated paste-impregnated gauze. Devitalized tissue, slough, viscous purulent drainage, debris, and other contaminants were cleared out of the tunneling areas and wound bed with each dressing change. By the end of the 10^{th} dressing change, the tunneling areas and wound bed appeared clean. Angiogenesis had begun with red colored, healthy connective tissue granulating through the wound. The wound dressing change schedule was changed to once every 2 days at this time to allow the granulation process to continue successfully. By the end of the fourth dressing on the new schedule, the tunneling areas were closing the open gaps, and merging towards the open area of the wound bed. Four more dressings continued on this schedule. By the end of the eighth dressing, tunneling areas had filled with growing collagen and closed. Uneven type III collagen was filling the visible area of the wound bed, and converting into smooth type I collagen. The wound bed was contracting in a healthy manner, and wound edges were approximating. Epithelial cells originating from edges and the wound bed were spreading over the collagen layer surface. The sacral wound dressing change schedule was set to once every 3 days at this time. Upon assessment, by the first dressing change on the new schedule, the moist texture to the wound surface had abated. The use of medicated paste was suspended at this time. A mixture of crushed ciprofloxacin and bacitracin zinc-polymyxin B sulfate powder was sprayed in the wound surface. A triple layer of 4″×4″ gauze pads was used to cover the wound, which was then covered by wound pads and secured with non-allergic tape. The wound was to be assessed every 2 days on the new wound dressing regimen. By the 2^{nd} evaluation, epithelial cells had formed a healthy scab that was completing the wound surface. Healthy smooth edges had inched more in the approximation process. By two more evaluations, the epithelial cells had fully scabbed the wound bed. The smooth, shiny, healthy sacral wound edges had formed a seamless line indicating the full maturation of the patient's sacral wound. The wound was left OTA at this time. Each of the three wounds (e.g., the wound of the right foot, the wound of the left foot, and the sacral wound) were monitored twice per week for two weeks. Then each wound was monitored once per week for 2 weeks, then once after 2 weeks. No wound reopened during this period of time. No wound had shown any irritation, nor any tendency towards infection.

### Example 11

A 75 year-old, male patient had infected wounds on the great toes of both feet. Both great toes were red, swollen, and in pain. Yellow, purulent drainage was present around the right great toenail, and purulent, yellow drainage was present all around the left great toenail as well. The drainage of the left great toe had a tinge of brownish color to it, with a foul odor permeating from both toes. The left great toenail was hanging to the skin only by the back end. The PCP ordered oral antibiotic amoxicillin (500 mg, tid, ×1 week) and wound care medication/supplies.

Daily dressing changes started with the wound of the right great toe using the following protocol: the wound of the right great toe was flushed with 0.9% normal saline (NS) (Nurse Assist, Inc., Reorder No. 6240), then patted dry with sterile 4″×4″ gauze pads. Bacitracin zinc-polymyxin B sulfate powder (about 3.3 g, POLYSPORIN® powder: polymyxin B sulfate 10,000 units/gram, bacitracin zinc 500 units/gram; Johnson & Johnson, NDC Code: 58232-4033) was thoroughly sprayed in the wound bed and gently applied under the right foot great toenail. A paste containing collagenase ointment (about 4 g, SANTYL® ointment: 250 units/gram; Smith+Nephew, NDC Code: 50484-010-30) and bacitracin zinc-polymyxin B sulfate powder (about 2.9 g) was formulated and laid on sterile 4″×4″ gauze pads. The resulting paste impregnated 4″×4″ gauze pads were then used to cover the wound bed. The wound dressing was next wrapped in KERLIX™ and stabilized with non-allergic tape.

The wound of the great toe of the left foot was flushed gently and thoroughly with 0.9% NS. Bacitracin zinc-polymyxin B sulfate powder (about 3.8 g) was thoroughly sprayed in the wound bed. A paste was formulated from collagenase ointment (about 4 g) and bacitracin zinc-polymyxin B sulfate powder (about 3.8 g). The formulated paste was laid on 4″×4″ gauze pads, which were then used to cover the wound of the great toe of the left foot. A double layer of clean dry 4″×4″ gauze pads was used to cover the paste impregnated layer, which was then wrapped with KERLIX™ and taped with non-allergic tape.

By the end of the fifth dressing, redness and swelling to the right foot had subsided. Drainage had diminished to minimal. The right great toenail had puffed up and looked ready to fall. By the end of the eighth dressing change, the right great toenail had fallen off. The ninth daily dressing was applied to the wound of the right great toe with the toe nail off for the first time. By the end of the second daily dressing change to this wound without the nail, the wound bed appeared clean. Vascularization started with healthy, new blood vessels proliferating in the wound bed. New, healthy connective tissue was growing in the wound bed. The wound dressing change schedule was set at this time for once every 2 days to allow the granulation process to proceed successfully. By the end of the third dressing on the new 2-day schedule, type III collagen was filling the wound bed. The wound edges appeared to be smoothening after the fall of the toenail, and appeared clean. The wound dressing change was set at this time for once every 3 days to allow the granulation process to finish. By the end of the first dressing on the 3-day schedule, uneven type III collagen appeared to be converting to type I, smooth collagen. The wound bed was also contracting, and the edges were approximating. Epithelial cells were spreading from the wound bed and edges, forming a smooth scab to cover the wound bed. The use of medicated paste was suspended at this time and the wound dressing protocol was amended as follows. Bacitracin zinc-polymyxin B sulfate powder was sprayed in the wound bed. A double layer of 4″×4″ gauze pads was used to cover the wound bed, which was gently wrapped with KERLIX™. The wound dressing was then taped with non-allergic tape. The wound was to be evaluated every 2 days using the new wound dressing protocol. By the second evaluation, the epithelial cells had formed a smooth, healthy layered scab that fully covered the wound bed. The wound edges were clean and healthily healed. The wound was left open to air (OTA) at this time.

The left great toenail had fallen off by the end of the third daily dressing change. The daily dressing change of the wound of the great toe of the left foot continued for six more times after the nail had fallen. By this time, the wound bed of the great toe of the left foot appeared very clean. Angiogenesis started as new healthy blood vessels appeared proliferating in the wound bed. Red colored, healthy new connective tissue was growing in the wound bed as well. The wound dressing change schedule for the wound of the great toe of the left foot was set at this time for once every 2 days. The same 0.9% NS cleansing measures, medication treatment regimen, and clinical wrapping procedures continued. The wound was to be thoroughly assessed upon each dressing change. By the end of the 6^{th} dressing change, healthy pink colored type III, uneven collagen appeared filling the wound bed of the great toe of the left foot. The wound bed was contracting well. The edges were smooth, clean and approximating closer. Epithelial cells were spreading from the wound edges and wound bed, forming a smooth layer to cover the surface of the wound of the great toe of the left foot. The wound dressing change schedule was changed to once every 3 days at this time. Similar 0.9% NS cleansing measures, medication treatment regimen, and clinical wrapping procedures were continued. By the end of the 2^{nd} dressing change, uneven collagen was converting into smooth layered type I collagen and was filling the wound bed. Epithelial cells had approximated and scabbed the wound. Edges continued to appear smooth and clean. The use of medicated paste was suspended at this time. Bacitracin zinc-polymyxin B sulfate powder was sprayed in the wound bed. A double layer of dry sterile 4″×4″ gauze pads was used to cover the wound bed, which was then wrapped with KERLIX™ and taped with non-allergic tape. By the third evaluation, type I, smooth layer collagen had filled the wound bed. Epithelial cells had formed a healthy smooth scab that was covering the wound bed. The wound edges appeared to have healed smoothly. The wound had fully matured and was left OTA at this time. Each wound was individually monitored twice per week×1 week, once per week×2 weeks, and finally, once every two weeks after the wounds healed. The wounds did not open during this period of time. Neither wound had shown any S/S of infection, nor any tendencies towards redness or irritation. By the eighteenth month, both great toenails had completely grown back.

### Example 12

A female patient had an abdominal resection related to stomach cancer. Upon examination, the surgical wound was extending from the left side of the patient's navel to 2 cm above the patient's left groin. The wound was fissured down the surgical incision. Viscous, dark-yellow drainage was leaking from the opened surgical incision. Other viscous, sticky, yellow drainage was mounted around the surgical incision, and to the edges. The surgical incision outer edges, edges inner sides, and the wound bed were pale colored and emitted a foul odor. Devitalized tissue, slough, and visible bacterial growth had mounted in the wound bed. Other portions of this same drainage burrowed itself in pockets under the surgical incision edges. The patient appeared pale and was relatively weak. The patient was discharged from the hospital the day before, and was admitted to the hospital because of septicemia. The PCP ordered a follow-up plan of care including medical supplies and morphine sulfate (20 mg, IR, PO).

The patient laid supine in bed with the head of bed (HOB) elevated 45 degrees. Patient was receiving O₂, 2 I/min/NC. 02 sat. was 95%. The prior wound dressing was removed, and the wound bed was flushed with 0.9% normal saline (NS) (Nurse Assist, Inc., Reorder No. 6240). The wound's edges were cleansed with 2″×2″ gauze pads, immersed in 0.9% NS. The wound bed and edges were patted dry with sterile 4″×4″ gauze pads. A mixture of crushed ciprofloxacin (2×500 mg, Aurobindo Pharma USA Inc., NDC Code: 658620077) and bacitracin zinc-polymyxin B sulfate powder (about 4.8 g, POLYSPORIN® powder: polymyxin B sulfate 10,000 units/gram, bacitracin zinc 500 units/gram; Johnson & Johnson, NDC Code: 58232-4033) was sprayed in the wound bed and upon the edges. A paste containing collagenase ointment (about 5 g, SANTYL® ointment: 250 units/gram; Smith+Nephew, NDC Code: 50484-010-30), crushed ciprofloxacin (2×500 mg), and bacitracin zinc-polymyxin B sulfate powder (about 3.8 g) was formulated and laid on sterile 4″×4″ gauze pads. The paste impregnated 4″×4″ gauze pads were used to cover the abdominal surgical wound. A triple layer of sterile 4″×4″ gauze pads was used to cover the paste impregnated layers. Sterile surgical wound pads were used to top the 4″×4″ gauze pads and were stabilized with non-allergic tape.

Daily wound dressing change following the above protocol was done for 12 changes. The wound was demonstrating improvement with each assessment upon each dressing change. Devitalized tissue, slough, contaminants, and other debris appeared to be clearing out of the wound bed. The wound edges were smoothening and clearing of any incision lines. New blood vessels were proliferating in the wound bed. New, healthy, red colored connective tissue was granulating the wound bed. The wound dressing schedule was set at this time for once every 2 days, still following the above protocol. By the end of the 8^{th} dressing change on the 2-day schedule, vascularization and granulation were proceeding successfully. Type III, uneven collagen was filling the wound bed. The wound bed was contracting. Smooth, clean, healthy surgical incision edges were approximating. Epithelial cells were clearly spreading to cover the wound surface. The wound dressing change schedule was set at this time for once every 3 days. By the end of the fourth dressing change on this schedule, type III, uneven collagen was converting to type I collagen and was filling the wound bed. The wound bed continued to contract. Heathy, healing edges were approximating closer towards maturation. The use of medicated paste was suspended at this time and the dressing protocol was amended as follows. A mixture of crushed ciprofloxacin and bacitracin zinc-polymyxin B sulfate powder was sprayed in the wound bed. A triple layer of sterile 4″×4″ gauze pads was used to cover the wound bed. Surgical wound pads were used to cover the sterile 4″×4″ gauze pads, which were then taped with non-allergic tape. The wound was to be evaluated every 2 days on the new antibiotic treatment/protocol. By the third evaluation, smooth collagen had filled the wound bed. Epithelial cells had formed a smooth layer covering the smooth collagen. Surgical incision edges were now only 2.5 cm apart from final closure. A mixture of crushed ciprofloxacin and bacitracin zinc-polymyxin B sulfate powder was sprayed in the wound. A triple layer of sterile 4″×4″ gauze pads was used to cover the wound bed and then taped with non-allergic tape. No wound pads were needed at this time. The wound was to be evaluated every 2 days. By the end of the second evaluation, the surgical incision edges had closed, forming a seamless, fully healed-surgical incision. The surgical wound had matured, and the wound was left open to air (OTA) at this time. The healed surgical wound was monitored twice per week for 2 weeks, then once per week for 3 weeks, and then once in 2 weeks. The wound did not open in this period. Six months later, the wound remained healed.

### Example 13

A 67 year-old, female patient had severe COPD and diastolic CHF. She was occasionally incontinent of urine and was using O₂ per nasal cannula at 2 liters per minute. The patient had frequent upper respiratory infections, leading to coughing out large amounts of phlegm. The patient was suffering from a deep tunneling sacral wound, and large blisters on both legs from below the knees to above the ankles. The patient had been suffering from these blisters and the resulting wounds for the last eight to nine years. The blisters were thought to be caused by Bullous pemphigoid, but they did not go away on their own as Bullous pemphigoid blisters do. The blisters did not develop in the patient's mouth nor the patient's mucous membranes. The doctors (PCPs and, dermatologists) previously administered sulfasalazine, diuretics, and antibiotics.

The tunneling sacral wound bed had dark, greenish, yellow, bronzy layers of necrotic tissue. A musty, slightly foul odor permeated from the wound. A foley catheter was inserted to aid with incontinence (size 14 FR foley catheter with 10 cc balloon, inserted with KY jelly lubricant). The catheter was to be changed every 4 weeks. The PCP ordered the necessary supplies for the wound treatment.

The wound of the right leg was about 2″ below the knee on the outer lateral aspect of the knee. The wound appeared to be resulting from the bursting of three blisters. The wound was circular in shape and was about 5.5 cm in diameter. The wound was reddish brown, having some contaminants/debris in the wound bed with tan, yellowish drainage. Drainage was seeping down the right leg. The patient complained of itching to the wound bed and leg. The wound was shallow.

The left leg had two wounds that averaged 5″ each in diameter. The first wound was below the knee on the anterior aspect, resulting from a cluster of ruptured blisters. The second wound was on the mid inner aspect of the left leg. Both wounds appeared in similar condition as the wound on the right leg. They were shallow, flaming red, brown, with some contaminants/debris in each wound bed. Seeping fluid moistened the wound beds and seeped down the leg. The seeping fluid caused irritation and itching to the left leg.

Treatment was started with the wound of the right leg. The wound bed was cleaned thoroughly with 0.9% normal saline (NS) (Nurse Assist, Inc., Reorder No. 6240). The wound bed was patted dry with sterile 4″×4″ gauze pads. Crushed ciprofloxacin (500 mg, Aurobindo Pharma USA Inc., NDC Code: 658620077) and bacitracin zinc-polymyxin B sulfate powder (about 2.9 g, POLYSPORIN® powder: polymyxin B sulfate 10,000 units/gram, bacitracin zinc 500 units/gram; Johnson & Johnson, NDC Code: 58232-4033) was sprayed thoroughly in the wound bed. A paste was formulated with collagenase ointment (about 3.5 g, SANTYL® ointment: 250 units/gram; Smith+Nephew, NDC Code: 50484-010-30), crushed ciprofloxacin (500 mg), and bacitracin zinc-polymyxin B sulfate powder (about 2.9 g). The formulated paste was laid on 4″×4″ gauze pads. The paste impregnated 4″×4″ gauze pads were used to cover the wound. A double layer of sterile, dry 4″×4″ gauze pads were used to cover the paste impregnated 4″×4″ gauze pads, which were then wrapped with KERLIX™ and taped with non-allergic tape.

The left foot was elevated, and the wound of the inner aspect was cleansed thoroughly with 0.9% NS. The wound was patted dry with sterile 4″×4″ gauze pads. A mixture of crushed ciprofloxacin (500 mg) and bacitracin zinc-polymyxin B sulfate powder (about 2.9 g) was thoroughly sprayed in the wound bed. A paste containing collagenase ointment (about 3.5 g), crushed ciprofloxacin (500 mg) and bacitracin zinc-polymyxin B sulfate powder (about 3.3 g) was formulated and laid on 4″×4″ gauze pads. The paste impregnated 4″×4″ gauze pads were then used to cover the wound. A double layer of dry sterile 4″×4″ gauze pads was then used to cover the paste impregnated layer. Two pieces of non-allergic tape held the dressing in place until the final wrapping with KERLIX™. The wound of the anterior aspect of the left leg was in similar condition to the one on the inner aspect. The same protocol was performed to the wound of the anterior aspect of the left leg. The medicated gauze pads of both wounds were wrapped with the same KERLIX™ sheet since they were on the same horizontal line. The resulting wound dressing was then taped with non-allergic tape.

There were about five blisters on the dorsal aspect of the left leg. The skin around the blisters was gently cleaned.

The sacral wound was flushed with 0.9% NS and patted dry with sterile 4″×4″ gauze pads. A mixture of crushed ciprofloxacin (2×500 mg) and bacitracin zinc-polymyxin B sulfate powder (about 7.6 g) was sprayed thoroughly in the wound bed and under the tunneling edges. A paste containing collagenase ointment (about 6.5 g), crushed ciprofloxacin (1.5×500 mg) and bacitracin zinc-polymyxin B sulfate powder (about 5.7 g) was formulated and laid on multiple 2″×2″ gauze pads and 4″×4″ gauze pads. The paste impregnated 2″×2″ gauze pads were packed under the tunneling edges. The paste impregnated 4″×4″ gauze pads were laid on top of the open wound bed. A triple layer of dry, sterile 4″×4″ gauze pads was used to cover the paste impregnated 4″×4″ gauze pads. Wound pads were then taped with non-allergic tape.

Blisters that were present or that developed on the patient's legs were to be monitored and assessed promptly. Daily dressing changes (following the above protocol) for the existing wound of the right leg, and the left leg two wound continued for seven days. Each dressing change showed significant improvement to each wound bed. Contaminant debris, moist yellow brownish, flaming red brown color, other infection and irritation signs and symptoms (S/S) were clearing and being removed gradually out of the wound bed. By the end of the 7^{th} dressing change, each wound bed appeared clean. Healthy, new blood vessels were proliferating in each wound bed. Healthy, red colored tissue was granulating in each wound bed. The wound dressing change was set to once every two days. Each wound assessment on the new 2-day schedule demonstrated positive improvement in the granulation process. By the end of the 5th 2-day dressing change, uneven collagen had almost filled each wound bed. Uneven type III collagen was converting into smooth layered, type I collagen. Wound beds were individually contracting. Epithelial cells were spreading from wound bed and edges to cover the collagen layer. The wound edges were smooth, clean and approximating well. The Wound dressing change schedule was set to once every 3 days at this time and the use of medicated paste was suspended from treatment of the wounds of both legs. The usual, 0.9% NS cleansing procedure was performed to each wound. A mixture of crushed ciprofloxacin and bacitracin zinc-polymyxin B sulfate powder was sprayed in each wound bed. The usual wrapping procedure with dry, sterile dressings, was followed. The wounds were to be individually evaluated every 2 days on the new only antibiotic treatment. By the 3^{rd} evaluation, all of the wound beds had fully contracted. Epithelial cells had formed a smooth, clean, healthy layer of scabbing over the smooth collagen that filled the wound bed. The smooth, shiny scab had merged with the smooth, cleanly healed edges, forming a continuous unification signaling maturation. Each wound was left open to air (OTA) at this time. Each wound was monitored and evaluated twice per week for 2 weeks. Then once per week for 2 weeks. Then once in 2 weeks. None of these wounds opened during this period of time. No blisters, no redness, nor any irritations appeared to the sites where those wounds were present.

Ten daily dressing changes were performed to the sacral wound using the procedures stated previously (e.g., cleansing with 0.9% NS, drying, applying antibiotics to the wound, applying medicated paste to the dressing, and using the same wound dressing protocol). The sacral wound demonstrated significant clinical improvement towards healing upon each assessment. The devitalized tissue, slough, sticky, multi-colored drainage, contaminants, and other debris were clearing out of the wound bed. By the end of the 10^{th} dressing change, the sacral wound bed appeared very clean. Vascularization started with new, healthy blood vessels proliferating in the wound bed. New, healthy, red colored connective tissue was granulating all over the wound bed. The sacral wound edges were smooth and clean. The dressing changing schedule for the sacral wound was set at this time for once every 2 days. The same 0.9% NS cleansing measures, medicated treatment regimen, and clinical wound wrapping procedures continued. By the end of the sixth dressing on the new schedule, vascularization and granulation appeared progressing successfully. Tunneling areas were closing and the wound was contracting. Smooth, clean edges were approximating. The same treatment plan was continued. By the end of the eighth dressing on the two-day schedule, the tunneling sacral wound area had closed. Uneven type III, pink, strong collagen was filling the wound bed. The wound bed continued to contract. Smooth, clean wound edges were approximating closer. Epithelial cells were spreading from the wound edges and wound bed, forming a smooth surface to cover the granulating collagen. By the end of the 10^{th} dressing change, uneven type III collagen was converting into smooth layered type I collagen. The wound bed was contracting, and the wound edges were approximating successfully. Epithelial cells continued to smoothly spread over the collagen surface. The sacral wound dressing change schedule was set to once every 3 days with the same treatment protocol (i.e., the same 0.9% NS cleansing measures, medicated treatment regimen, and clinical wound wrapping procedures). By the end of the 2^{nd} dressing upon the new 3-day schedule, smooth layered, type I collagen had filled the wound bed. Epithelial cells had spread over the smooth collagen layer, forming a smooth surface covering the wound bed. The wound edges continued to unify with the spreading wound surface. Five moist spots, nickel and dime-sized, appeared towards the lateral edges. The use of medicated paste was suspended at this time. A mixture of crushed ciprofloxacin and bacitracin zinc-polymyxin B sulfate powder was sprayed in the wound bed. A triple layer of sterile 4″×4″ gauze pads was used to cover the wound bed. Wound pads were placed on top of the 4″×4″ gauze pads, which were then taped with non-allergic tape. The wound was to be evaluated every 2 days upon the new treatment protocol. By the 3^{rd} evaluation, the wound surface appeared shiny and clean. Epithelial cells had formed a healthy scab over the wound bed. The healed scab had smoothly merged with the approximating edges and formed a seamless unification for a matured wound. The wound was left OTA at this time, and was monitored 2 times per week for the first 2 weeks, then 1 time per week for 2 weeks and 1 time in 2 weeks. The sacral wound did not reopen during this period of time, or show any signs of any irritations.

By the end of the 3^{rd} dressing change on the 2-day schedule treating the wounds on both legs, the five engorged blisters on the dorsal aspect of the left leg burst, forming a wider wound about 7 cm in diameter and about 1.5 cm deep. The new bursting blisters of the dorsal aspect of the left leg had a similar color to the other leg wounds when they started. The color was flaming reddish, brown. A similar 0.9% NS cleansing measures, wound medicated treatment, and clinical wound procedure was followed in treating this wound: the wound was thoroughly flushed with 0.9% NS to remove contaminants, debris, and skin pieces. The wound was patted wound dry with sterile 4″×4″ gauze pads. Bacitracin zinc-polymyxin B sulfate powder (about 5.7 g) and crushed ciprofloxacin (1.5×500 mg) were sprayed thoroughly in the wound bed. A paste containing collagenase ointment (about 5 g), bacitracin zinc-polymyxin B sulfate powder (about 5.7 g), and crushed ciprofloxacin (1.5×500 mg) was formulated and laid on 4″×4″ gauze pads. The paste impregnated 4″×4″ gauze pads were used to cover the wound bed. A double layer of sterile, dry gauze was used covered the medicated layer. The resulting dressing was wrapped with KERLIX™ and taped with non-allergic tape. The wound dressing change was to be done daily for this new wound and was to be assessed upon each dressing change. By the end of the 6^{th} daily dressing change, the wound bed appeared very clean. Vascularization had started with new, healthy blood vessels proliferating in the wound bed. Red colored connective tissue was granulating in the wound bed as well. The wound appeared to be contracting well. The edges were smooth and clean. The wound dressing change was set to once every 2 days at this time. Similar 0.9% NS cleansing measures, medicated treatment regimen, and clinical wound wrapping procedures were continued. By the end of the 4th 2-day dressing change, the granulation process was progressing successfully. Progress was demonstrated by strong, pink, healthy uneven type III collagen that filled the wound bed. The wound bed continued to contract. The wound edges continued to smoothen and approximate closer. Epithelial cells had started spreading from the wound bed, and the wound formed a healthy surface covering the granulating tissue. The wound dressing change schedule was set to once every 3 days. Two dressings were performed on the 3-day schedule. By the end of the 2^{nd} dressing change on the 3-day schedule, uneven collagen was filling the wound bed. Uneven collagen was converting into smooth layered type I collagen. Epithelial cells were spreading wider, forming a healthy scab over the wound bed. Clean, smooth edges were approximating closer towards the scabbing wound surface. The use of medicated paste was suspended at this time. Bacitracin zinc-polymyxin B sulfate powder was sprayed in the wound bed. A double layer of 4″×4″ gauze pads was used to cover the wound bed. The resulting dressing was wrapped with KERLIX™ and taped with non-allergic tape. The wound was to be evaluated every 2 days on the new treatment protocol. By the 2^{nd} evaluation, smooth collagen had filled the wound bed. Epithelial cells had completed forming a smooth surface covering the collagen layer. The smooth epithelial surface had scabbed the wound bed. The nascent scab had merged with the smooth, clean healed edges. The merger had formed a seamless unification for the healed site and the wound was left OTA at this time. The dorsal aspect left leg wound was monitored and evaluated similarly to the other leg wounds. This wound did not reopen. Abnormal S/S did not appear to this wound site during this time. By the end of the 2^{nd} dressing change of the wound of the, dorsal aspect of the left leg on the 2-day schedule, three new blisters were appearing on the lower inner aspect of the right leg. By the end of the 2^{nd} dressing change on the 3-day schedule for the wound of the dorsal aspect of the left leg, three clustered blisters of the lower inner aspect of the right leg had burst, forming a new wound. The wound surface was 3.75 cm×5 cm and 1.6 cm deep. A similar treatment was utilized to treat the previous wounds.

The daily dressing change involved flushing the wound with 0.9% NS, spraying the wound bed with bacitracin zinc-polymyxin B sulfate powder (about 3.8 g) and crushed ciprofloxacin (500 mg)), paste formulated from collagenase ointment (about 3.5 g), bacitracin zinc-polymyxin B sulfate powder (about 2.9 g), and crushed ciprofloxacin (500 mg), and the usual clinical wound wrapping procedures. Daily dressing changes were performed for 6 days. By the end of the 6th daily dressing change, the wound appeared clean, with vascularization and granulation starting in a healthy manner. The wound dressing schedule was set to once every 2 days with a similar treatment process. By the 4th wound dressing change on this schedule, granulation had continued successfully. Uneven collagen almost filled the wound bed. The wound bed was contracting. The wound edges were smooth and clean. Healthy epithelization had started. The wound dressing change schedule was set to once every three day at this time. This was to allow the granulation process to continue successfully. By the end of the first dressing change on the new 3-day schedule, type III collagen was converting into smooth layered type I collagen, filling the wound bed. The wound continued to contract properly. Smooth, clean edges were approximating closer. Epithelial cells had formed a smooth surface to cover the wound bed. The use of medicated paste was suspended at this time. Bacitracin zinc-polymyxin B sulfate powder was sprayed in the wound bed. A double layer of 4″×4″ gauze pads was used to cover the wound bed. The resulting dressing was wrapped with KERLIX™. The wound dressing was then taped with non-allergic tape. By the 2^{nd} 3-day schedule evaluation, the wound bed had fully contracted. Epithelial cells had formed a complete surface covering the wound bed. Healthy clean smooth edges approximated closer towards the wound surface. Bacitracin zinc-polymyxin B sulfate powder was sprayed in the wound bed. The wound was covered with a double layer of 4″×4″ gauze pads, which was then wrapped with KERLIX™ and taped with non-allergic tape. The wound was to be evaluated every 2 days at this time. By the end of the 2^{nd} evaluation, smooth layered type I collagen had filled the wound bed. Epithelial cells had formed a smooth surface that was scabbing the collagen layer of the wound bed. The scab merged with the healed wound edges, forming a seamless unified surface indicative of a correctly healed wound. The wound was left OTA at this time. The wound was monitored and evaluated twice per week for 2 weeks, once per week for 2 weeks, then once in 2 weeks. The wound did not open, nor did it show any S/S of infection, redness to the wound site, or adjacent skin areas.

Eleven months passed since the patient was discharged from home health care. The patient never had a new blister, a new sore or a new wound on her legs or sacral area nor anywhere on her body all this time.

### Example 14

A male patient had an open, necrotic sacral wound. The wound was 12 cm×8 cm and was 3 cm deep. Devitalized tissue, slough, contaminants, debris, and other growths inhabited the wound bed. The slough in the wound was stringy yellow meshed with a map of multi-colors. A foul odor was permeating from the wound. The skin area surrounding the wound area had a relatively normal color, suggesting adequate circulation and proper skin perfusion outside the wound boundaries. The pain caused by the sacral wound was more than 10 out of 10, as perceived by the patient. The patient had another wound to the lower anterior aspect of the right leg. This wound was circular with a diameter of about 8.5 cm and depth of 1.625 cm. The wound appeared to be wet and gangrenous. The wound produced a crackling sound when the tissue was pressed. The patient was in remission from an early skin cancer to the face and neck. The patient had type II diabetes, a history of stomach ulcers associated with acid reflux, a mild case of diverticulitis, and a history of managed asthma with Symbicort and Ventolin inhalers.

The PCP ordered Clindamycin (450 mg, po, 3×s a day for 10 days). The patient was laid to his left side to clean and dress the sacral wound. The wound was flushed with 0.9% normal saline (NS). A mixture of bacitracin zinc-polymyxin B sulfate powder (about 5.7 g, POLYSPORIN® powder: polymyxin B sulfate 10,000 units/gram, bacitracin zinc 500 units/gram; Johnson & Johnson, NDC Code: 58232-4033) and crushed ciprofloxacin (2×500 mg, Aurobindo Pharma USA Inc., NDC Code: 658620077) was thoroughly sprayed in the wound bed, reaching the tunneling areas, and covering the edges of the wound. A paste containing collagenase ointment (about 6 g, SANTYL® ointment: 250 units/gram; Smith+Nephew, NDC Code: 50484-010-30), bacitracin zinc-polymyxin B sulfate (about 5.7 g) and crushed ciprofloxacin (1.5×500 mg) was formulated and placed on 4″×4″ gauze pads. The paste impregnated gauze was used to cover the wound. A triple layer of dry, sterile 4″×4″ gauze pads was used to cover the paste impregnated 4″×4″ gauze pads, which were then covered with wound pads. The wound pads were subsequently taped with non-allergic tape.

The right foot was placed on a pillow to elevate the leg and manipulate the wound from all sides safely. The leg wound was flushed with 0.9% NS. Similarly, a paste of collagenase ointment (about 5 g), bacitracin zinc-polymyxin B sulfate (about 4.8 g) and crushed ciprofloxacin (500 mg) was formulated and laid on 4″×4″ gauze pads which covered the leg wound. The resulting dressing was wrapped gently with KERLIX™ and taped with non-allergic tape. The dressing changes for the sacral wound and wound of the anterior aspect of the right leg were to be done daily for the foreseen period.

The daily dressing change to the wound of the anterior aspect of the right leg was done for eleven consecutive days. By the end of the 7^{th} dressing change, the crackling sound coming from pressing on the wound of the anterior aspect of the right leg tissue had dissipated. By the end of the 9^{th} wound dressing change, the sloughing, necrotic tissue had almost completely cleared from the wound bed. By the end of the 11^{th} dressing change, the wound bed of the anterior aspect of the right leg appeared clean, and pink in color. Vascularization had started with new, healthy blood vessels proliferating in the wound bed. New, red connective tissue was granulating in the wound bed as well. The dressing change schedule for the wound of the right leg was set to once every 2 days at this time. The wound was to be assessed upon each dressing change. Vascularization and granulation appeared progressing successfully upon each assessment. Healthy, pink, thick, uneven type III collagen continued filling the wound bed. The wound bed was contracting well. The wound edges were approximating properly. By the end of the 6^{th} dressing change, collagen appeared to almost fill the wound bed. Uneven collagen was converting into type I, smooth layered collagen. Epithelial cells were spreading from the wound edges and the wound bed, forming a smooth surface to cover the collagen layer. Smooth, clean wound edges were approximating closer. The wound dressing change schedule was changed to once every 3 days at this time. By the end of the first dressing change, the wound appeared to have completed the contraction process. Smooth layered collagen had filled the wound bed. Epithelial cells formed a smooth surface that had almost completely covered the wound bed. The use of medicated paste was suspended at this time. A mixture of crushed ciprofloxacin and bacitracin zinc-polymyxin B sulfate powder was sprayed in the wound bed. The wound bed was then covered with a double layer of 4″×4″ gauze pads. The dressing was further wrapped with KERLIX™ and taped with non-allergic tape. The wound was to be evaluated every 2 days following the new wound medicating/dressing protocol. Upon the 2^{nd} evaluation, epithelial cells had healthily and fully scabbed the wound bed. The shiny, smooth, healthy scab had merged with the smooth, cleanly healed wound with approximating edges. The wound was left open to air (OTA) at this time. The wound was monitored and assessed twice per week×1 week, then once per week×2 weeks, and then once in 2 weeks. The wound of the anterior aspect of the right leg did not open during this period of time. The wound did not show any S/S of redness, irritation, nor infection during this period of time.

The sacral wound daily dressing changes went on for 12 days (using the medicated paste protocol above). Upon assessment on each dressing change, the sacral wound demonstrated significant improvement. Devitalized tissue, slough, contaminants, debris, and other growths that inhabited the wound were cleared out. Similar to wound of the right leg, 0.9% NS cleansing measures, medicated paste regimen, and clinical wound wrapping procedures were performed in treating the sacral wound. By the end of the 12^{th} daily dressing change, the sacral wound bed and tunneling areas of the wound appeared very clean. New, healthy blood vessels were proliferating in the wound bed. New, healthy red colored tissue was granulating in the wound bed. The sacral wound dressings change was changed to once every 2 days at this time using the same treatment protocol. By the end of the 6^{th} dressing change, the tunneling areas were filling with collagen and were closing towards the open wound bed. Uneven collagen was filling the visible open area of the wound bed as well. Uneven, thick collagen was converting to type I collagen. The wound bed contraction continued as well. The wound edges were approximating properly. Epithelial cells were spreading from the wound bed, and the wound edges were forming a smooth surface to cover the collagen layer. The dressing change schedule for the wound was set to once every 3 days at this time. By the end of the 2^{nd} dressing change, type I, smooth collagen had filled the wound bed and tunneling areas. The tunneling areas had fully closed and merged with the visible open wound bed. Epithelial cells had formed a smooth, shiny layer covering the wound bed. Clean, smooth, healthy wound edges had approximated closer. The use of medicated paste was suspended at this time. A mixture of crushed ciprofloxacin and bacitracin zinc-polymyxin B sulfate powder was sprayed in the wound bed and was then covered by a triple layer of 4″×4″ gauze pads. The resulting dressing was then covered with wound pads and taped with non-allergic tape. The wound was to be evaluated every 2 days on the new only antibiotic treatment. Upon the 3^{rd} evaluation, healthy, strong, pink, smooth layered type I collagen had filled the wound bed. Epithelial cells had formed a shiny, healthy, smooth surface covering the wound bed. The clean, smooth, healthy approximating edges had formed a complete wound maturation. The wound was left OTA at this time. Four months after treatment, the sacral wounds and wounds of the right leg had not reopened and were healed.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A topical composition for treating a wound in a subject comprising at least first and second layers, wherein:
(a) the first layer comprises a powder, wherein the powder comprises 25-95% by total weight bacitracin zinc-polymyxin B sulfate powder comprising 100-1,000 units bacitracin per gram and 5,000-15,000 units polymyxin B per gram and 5-75% by total weight ciprofloxacin; and
(b) the second layer comprises an ointment, wherein the ointment comprises:
(i) an admixture comprising 40-80% by total weight collagenase ointment comprising 100-500 units collagenase per gram, 4-50% by total weight ciprofloxacin powder, and 20-50% by total weight bacitracin zinc-polymyxin B sulfate powder comprising 100-1,000 units bacitracin per gram and 5,000-15,000 units polymyxin B per gram; or
(ii) an admixture comprising 40-80% by total weight collagenase ointment comprising 100-500 units collagenase per gram, and 20-60% by total weight bacitracin zinc-polymyxin B sulfate powder comprising 100-1,000 units bacitracin per gram and 5,000-15,000 units polymyxin B per gram.

2. The topical composition of claim 1, wherein the first and/or second layer further comprises 4-30% by total weight methicillin sodium powder.

3. The topical composition of claim 1, wherein the first and/or second layer further comprises 4-30% by total weight ceftaroline fosamil powder.

4. The topical composition of claim 1, wherein the admixture of (b)(ii) further comprises 4-30% by total weight ceftazidime sodium carbonate dry powder comprising 110-120 mg sodium carbonate per gram of ceftazidime.

5. A method for treating a wound in a subject comprising topically applying the composition of claim 1 to the wound, wherein the first layer is applied to the wound and the second layer is applied over the first layer.

6. The method of claim 5, wherein, prior to application of the composition, the wound is cleaned with 0.05-0.10% normal saline (NS).

7. The method of claim 5, wherein the second layer comprises a gauze that is impregnated with the ointment, and wherein the gauze is applied over the first layer.

8. The method of claim 7, further comprising covering the gauze with an additional layer of gauze, KERLIX™, and/or a wound pad.

9. The method of claim 8, wherein the additional layer of gauze, KERLIX™, and/or wound pad is secured to the subject with tape.

10. The method of claim 5, wherein the wound is an external ulcer or a pressure ulcer, a surgical wound, is caused by or results from diabetes or systemic lupus erythematosus (SLE), or is infected, or wherein the wound comprises gangrene.

11. The method of claim 10, wherein the external ulcer is a diabetic foot ulcer or the pressure ulcer is a bed sore.

12. The method of claim 10, wherein the infected wound comprises methicillin-resistant Staphylococcus aureus (MRSA) or Pseudomonas aeruginosa.

13. The method of claim 5, wherein the method comprises topically applying the composition to the wound one or more times daily, weekly, biweekly, or monthly.

14. The method of claim 13, wherein the topical application occurs one or more times every one, two, three, four, five, six, or seven days.

15. The method of claim 5, wherein the topical application occurs for 1 to 52 weeks or more.

16. The method of claim 5, wherein the subject is a human.

17. The method of claim 5, wherein the first and/or second layer of the composition further comprises 4-30% by total weight methicillin sodium powder and/or 4-30% by total weight ceftaroline fosamil powder.

18. The method of claim 5, wherein the admixture of (b)(ii) further comprises 4-30% by total weight ceftazidime sodium carbonate dry powder comprising 110-120 mg sodium carbonate per gram of ceftazidime.
